(19) United States
(12) Patent Application Publication
(10) Pub. No.: US 2026/0076823 A1
(43) Pub. Date: Mar. 19, 2026

(54) Cooling and Refrigeration Based on Vacuum-Driven Water Evaporation

(71) Applicant: CleanCold Technologies LLC, Andover, MA (US)

(72) Inventors: John P. O’Connor, Andover, MA (US); Cindy A. O’Connor, Andover, MA (US); Ellen E. Sheets, Boston, MA (US)

(73) Assignee: CleanCold Technologies LLC, Andover, MA (US)

(21) Appl. No.: 19/336,989

(22) Filed: Sep. 23, 2025

Related U.S. Application Data
(63) Continuation of application No. 18/377,808, filed on Oct. 8, 2023.
Application No. 18/377,808 is a continuation of application No. 16/845,943, filed on Apr. 10, 2020, now Pat. No. 11,779,488.
(60) Provisional application No. 62/832,257, filed on Apr. 10, 2019.
(60) Provisional application No. 62/859,767, filed on Jun. 11, 2019.
(60) Provisional application No. 62/880,189, filed on Jul. 30, 2019.
(60) Provisional application No. 62/969,876, filed on Feb. 4, 2020.
(60) Provisional application No. 63/698,020, filed on Sep. 23, 2024.
(60) Provisional application No. 63/704,507, filed on Oct. 7, 2024.
(60) Provisional application No. 63/729,407, filed on Dec. 8, 2024.
(60) Provisional application No. 63/791,676, filed on Apr. 20, 2025.

(51) Int. Cl. A61F 7/00; A61B 18/00; A61B 18/02; A61F 7/12; F25B 19/04; F25B 47/00

(52) U.S. Cl. CPC A61F 7/0085; A61B 18/0218; A61F 7/12; F25B 19/04; F25B 47/006; A61B 2018/00029; A61B 2018/00464; A61B 2018/00494; A61B 2018/00577; A61F 2007/0065

(57) ABSTRACT

Apparatus for cooling an object, space, or tissues of a patient. A vacuum chamber is designed to be placed in thermal contact with the object or space to be cooled, or against a patient to be treated. A water sprayer is configured to spray water into the vacuum chamber or against a cooling wall of the chamber. A vacuum pump and computer control are designed to maintain vacuum below ambient pressure in the vacuum chamber sufficient to cause accelerated evaporation of the water and cooling to a temperature desired for cooling of the object, space, or patient.

This application claims benefit of U.S. Provisional application Ser. No. 63/791,676, filed Apr. 20, 2025, titled Cooling and Refrigeration Based on Vacuum-Driven Water Evaporation; claims benefit of U.S. Provisional application Ser. No. 63/729,407, filed Dec. 8, 2024, titled Cooling and Refrigeration Based on Vacuum-Driven Water Evaporation; claims benefit of U.S. Provisional application Ser. No. 63/704,507, filed Oct. 7, 2024, titled Cooling and Refrigeration Based on Vacuum-Driven Water Evaporation; claims benefit of U.S. Provisional application Ser. No. 63/698,020, filed Sep. 23, 2024, titled Cooling and Refrigeration Based on Vacuum-Driven Water Evaporation; is a continuation of U.S. application Ser. No. 18/377,808, filed Oct. 8, 2023, titled Cooling and Refrigeration Based on Vacuum-Driven Water Evaporation; which is a continuation of U.S. application Ser. No. 16/845,943, filed Apr. 10, 2020, titled Cooling and Refrigeration Based on Vacuum-Driven Water Evaporation, now issued as U.S. Pat. No. 11,779,488; the '943 application claims benefit of U.S. Provisional application Ser. No. 62/969,876, filed Feb. 4, 2020, titled Cooling and Refrigeration Based on Vacuum-Driven Water Evaporation, now expired; the '943 application claims benefit of U.S. Provisional application Ser. No. 62/880,189, filed Jul. 30, 2019, titled Cooling and Refrigeration Based on Vacuum-Driven Water Evaporation, now expired; the '943 application claims benefit of U.S. Provisional application Ser. No. 62/859,767, filed Jun. 11, 2019, titled Cooling and Refrigeration, now expired; the '943 application claims benefit of U.S. Provisional application Ser. No. 62/832,257, filed Apr. 10, 2019, titled Cooling of Tissue, now expired. Each of the preceding is incorporated by reference.

### BACKGROUND

This application relates to cooling and refrigeration based on vacuum-driven water evaporation.

### SUMMARY

In general, in a first aspect, the invention features a method. A vacuum chamber is placed against tissues of the patient for which cooling is desired for medical treatment. Water is sprayed from a mister into the vacuum chamber or against a cooling wall of the vacuum chamber. Vacuum is maintained in the vacuum chamber sufficient to cause accelerated evaporation of the water and cooling to a temperature desired for cooling of tissues of the patient.

In general, in a second aspect, the invention features apparatus for treating a patient. A vacuum chamber has a cooling wall designed to be placed against tissues of the patient for which cooling is desired for treatment. A water sprayer is designed to spray water into the vacuum chamber. A vacuum pump and computer control 490 are designed to maintain vacuum in the vacuum chamber sufficient to cause accelerated evaporation of the water and cooling to a temperature desired for cooling of tissues of the patient.

In general, in a third aspect, the invention features a method of cooling an object or space. A vacuum chamber is placed in thermal contact with tissue, an object, or space to be cooled. Water is sprayed from a mister into the vacuum chamber. Vacuum below room ambient pressure is maintained in the vacuum chamber sufficient to cause accelerated evaporation of the water and cooling of the tissue, object, or space to a desired temperature below its ambient temperature.

In general, in a fourth aspect, the invention features apparatus for cooling an object or space. A vacuum chamber has a cooling wall designed to be placed against the object or space to be cooled. A water sprayer is configured to spray water into the vacuum chamber or against the cooling wall. Apparatus is designed to maintain vacuum below ambient pressure in the vacuum chamber sufficient to cause accelerated evaporation of the water and cooling of the cooling wall to a temperature desired for cooling of the object or space.

Embodiments of the invention may include one or more of the following features. These features may be used singly, or in combination with each other. The cooling wall of the vacuum chamber may be a cylinder with a vertical axis. The water sprayer may be located on the cylinder axis. An enclosure may surround the vacuum chamber. A space between the outer enclosure and vacuum chamber may be designed as a water jacket. The water jacket may provide flow of water through a space between the enclosure and vacuum chamber. The water flow may carry cooling to the tissue, object, or space by convection. The computer control may be programmed to cycle a water pump and/or the vacuum pump to maintain a temperature for cooling of the tissue, object, or space. A conical chamber may lie between the water sprayer and the cylindrical cooling wall cylinder. A chamber in gas communication with the cylindrical vacuum chamber may be of volume to contain air to dissolve water vapor. The cooling wall cylinder may be formed of aluminum or an aluminum alloy. The cooling wall cylinder may be roughened or otherwise treated to increase surface tension with droplets of cooling water. The computer control may be programmed to cycle the vacuum pump on-off or to cycle speed of the vacuum pump to reduce icing. The computer control may be programmed to cycle the vacuum pump on-off or to cycle speed of the vacuum pump to maintain pressure in the vacuum chamber near the pressure corresponding to the temperature for cooling of the tissue, object, or space. The computer control may be programmed with control parameters optimized using multi-parameter factorial experiment optimization. The apparatus may have a second vacuum chamber. The computer control may be programmed to cycle draw of the vacuum pump between the two chambers. The vacuum chamber may be formed as open vacuum bell open on a side to be sealed to the tissue, object, or space. The vacuum chamber may be formed as an enclosed volume having a cooling wall at one side, and the cooling wall may be designed to be placed in physical contact with the object, space, or tissues of the patient. The vacuum chamber may be formed as a vacuum bell sealed to a flat thermally conductive platen, and the platen may be designed to be placed in thermal contact with the tissue, object, or space. The platen may be coated with a non-metallic nonstick release material designed to prevent adhesion and resultant tissue damage resulting from freezing of the tissue to be cooled or water between the tissue and the platen. The water may hold in solution an electrolyte chosen to depress freezing point of the water to a desired temperature. The vacuum chamber may include one or more thermal sensors. Vacuum may be controlled by a computer control programmed to obtain thermal data from the one or more thermal sensors, and to control one or more of water flow rate, vacuum pressure, and electrolyte solution, to control for a desired temperature and/or rate of cooling, and/or to correct for various confounders in thermal flow into the vacuum chamber. The tissue to be cooled may be tissue of a human patient, such as adipose tissue, skin, cancerous tissue, malignant cells, undesired benign cells that are selectively sensitive to cold, or goblet cells. The cooling treatment may be for purposes of disrupting fat, to reduce pain, to lighten skin and/or to reduce hypopigmentation, or to ablate undesired tissue. The tissue may be skin, in the gastrointestinal tract, or in the respiratory tract. The cooling may be designed to disrupt undesired cells. The space to be cooled may be an enclosed space to be cooled to refrigeration or freezing temperatures. The space to be cooled may be a room to be cooled to air conditioning temperatures.

The above advantages and features are of representative embodiments only, and are presented only to assist in understanding the invention. It should be understood that they are not to be considered limitations on the invention as defined by the claims. Additional features and advantages of embodiments of the invention will become apparent in the following description, from the drawings, and from the claims.

### DESCRIPTION OF THE DRAWINGS

FIGS. 1A, 2, 3B-3D, 4B, 4C, and 4F, are perspective views, partially cut away, of cooling apparatus.

FIGS. 1B, 1C, 4D, 4E, 4G, and 5A-5C are schematic views of a cooling apparatus.

FIG. 3A is a section view of a body lumen being treated with a catheter.

FIG. 4A is a section view of a cooling apparatus.

### DESCRIPTION

The Description is organized as follows.
I. Introduction and overview
II. Apparatus for vacuum-driven evaporative cooling
   II.A. Vacuum bell sealing against the skin
   II.B. Electrolyte solutions as coolant
   II.C. Replacement water
III. Application as a Refrigeration or Air Conditioning System
   III.A. Application as a refrigeration or air conditioning system with concurrent humidification of an external region
   III.B. Prevention of water freezing
   III.C. Water droplet control
IV. Cooling for medical and therapeutic applications
   IV.A. Cryolipolysis
   IV.B. Vacuum bell sealing against the skin
   IV.C. Cooling for analgesia
   IV.D. Cooling for hypopigmentation
   IV.E. Cooling for pharmaceutical transportation cold chain
   IV.F. Cooling for tissue ablation within the body
V. Additional spray improvements
   V.A. Apparatus
   V.B. Operation
      V.B.1. A first cycle
      V.B.2. A second cycle that reduces cycling from vacuum to ambient pressure
      V.B.3. Computer control
   V.C. Dual chambers
   V.D. Control of bleed gas and water spray and multi-stage pump
VI. Embodiments

### I. INTRODUCTION AND OVERVIEW

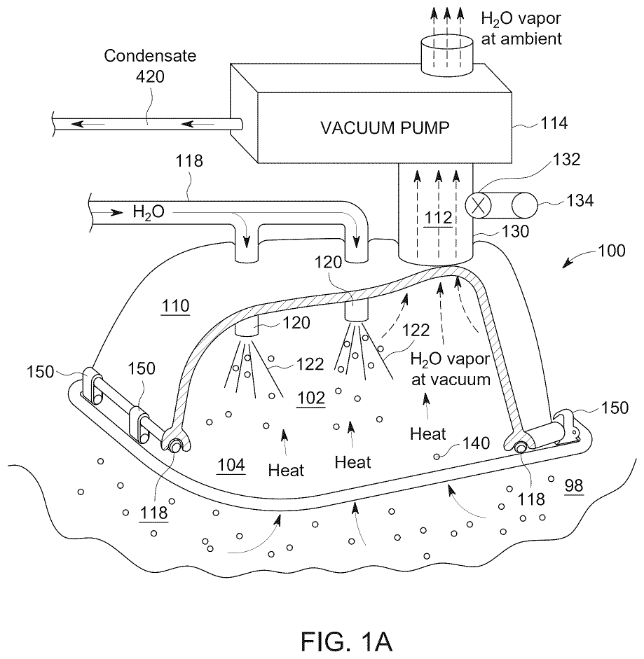

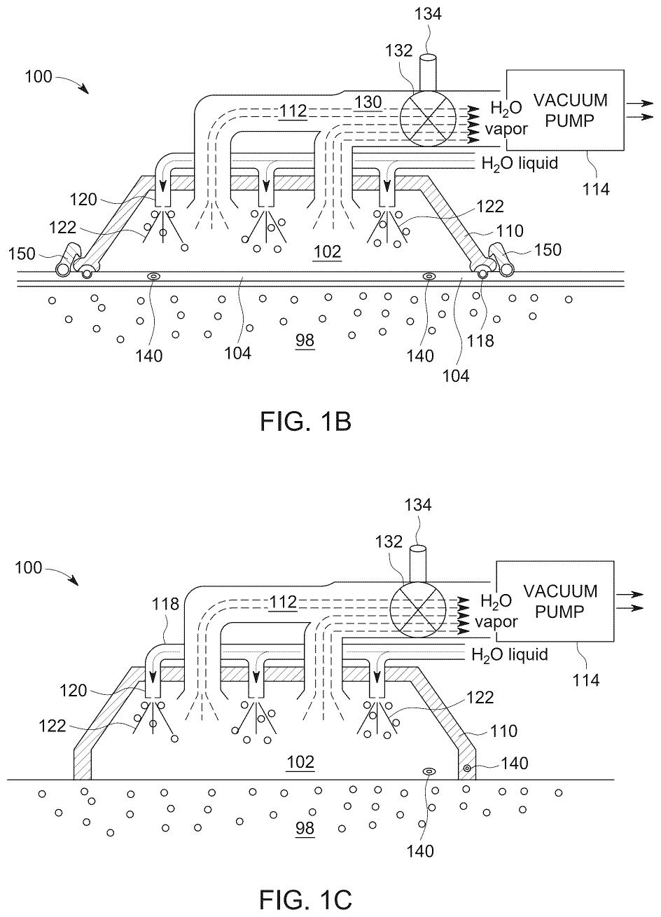

Referring to FIGS. 1A and 1B, a vacuum cooling apparatus 100 may be used to cool a selected region 98 by vaporization of a liquid, especially a liquid with a high enthalpy of vaporization, for example, water. Vaporization, and thus cooling, may be accelerated and controlled by applying vacuum above the liquid. A vacuum chamber 102 may be formed as an enclosed volume 102 with a conductive side 104 for providing cooling to the selected region, and apparatus 100 may be arranged to effect evaporation or sublimation of the water against that thermally-conductive side 104. A vacuum may be drawn in vacuum chamber 102 that drives evaporation or sublimation of the liquid. The energy required to provide the heat of vaporization is drawn through thermally-conductive side wall 104 of vacuum chamber 102, thus lowering the temperature of conductive side 104, which in turn cools whatever 98 is on the other side of that conductive wall. Vacuum in chamber 102 drives the vaporization of the coolant, and then the heat of vaporization is pulled from the subject 98 to be cooled.

Convection, such as a column of moving air, may be used to move the cooling to desired regions. With an appropriate electrical and vacuum pump 114 arrangement, the device may be controlled to generate a controlled and precise drop in temperature of the conductive plate and hence of region 98 to be cooled to a desired level.

Apparatus 100 may enable thermal contact between the cooled conductive element with tissue to provide desired cooling of the tissue to specified temperatures. Selected regions 98 of tissue of a body may be cooled for various diagnostic or therapeutic purposes.

Referring to FIG. 1C, vacuum chamber 102 may be formed by a bell that seals against the skin 98, and apparatus 100 may be arranged to effect evaporation at the surface of the skin. A vacuum is drawn in vacuum chamber 102 that drives evaporation of the liquid. The energy required to provide the heat of vaporization is drawn from the tissue. As a result, energy is removed from the tissue and the tissue is cooled. With an appropriate electrical and vacuum pump 114 arrangement, computer control 490 may control the device to generate a controlled and precise drop in temperature of tissue 98. In this way, certain tissue, such as adipose tissue, may be disrupted to provide a pathway for reabsorption of the tissue by the body and elimination of such tissue. In cases where there is no platen 104 to carry thermal sensors 140, sensors may be placed on the surface of the object 98 to be cooled, or in the wall of vacuum bell 110, or elsewhere.

This refrigeration/cooling apparatus may be used for a variety of purposes: food storage, medical therapies, pharmaceutical transportation cold-chain, and the like.

### II. APPARATUS FOR VACUUM-DRIVEN EVAPORATIVE COOLING

Referring to FIGS. 1A and 1B, vacuum-driven evaporative cooling may provide desired cooling in a rapid and controlled manner. A vacuum bell 110 may placed in contact with a thermally conductive platen 104, and sealed to platen 104 at edges through the use of an O-ring or similar seal 118 around edge of thermally-conductive platen 104. Seal 118 prevents or reduces leaking of air into volume 102 enclosed between platen 104 and vacuum bell 110, and may provide thermal insulation between thermally conductive platen 104 and vacuum bell 110.

A spray device 120 may provide a spray or mist of liquid 122, preferably a liquid with a high specific heat of vaporization such as water, that is applied to the outer surface of thermally-conductive platen 104 within volume 102 between platen 104 and vacuum bell 110. A vacuum may be drawn into volume 102 by a suitable vacuum pump 114 connected to vacuum bell 110 through a gas conductive region such as a hose or pipe 130 through a three-way valve 132. As a result of the vacuum within the enclosure, liquid 122 within volume 102 will vaporize and become a gas, that is, water vapor if the liquid selected is water. The energy to vaporize the liquid is removed from conductive platen 104. The energy of the platen 104 is lowered and hence the temperature of the platen is lowered. This in turn cools tissue or region 98. Three-way valve 132 is used to connect to vacuum pump 114 or air inlet 134 to provide either vacuum suction from vacuum pump 114 or venting of chamber 102 using air inlet 134.

Water may be selected as the liquid to be misted and then vaporized within chamber 102. Due to the high vaporization energy of water (2,256 kilojoules per kilogram), a significant amount of heat may be removed from thermally conductive platen 104.

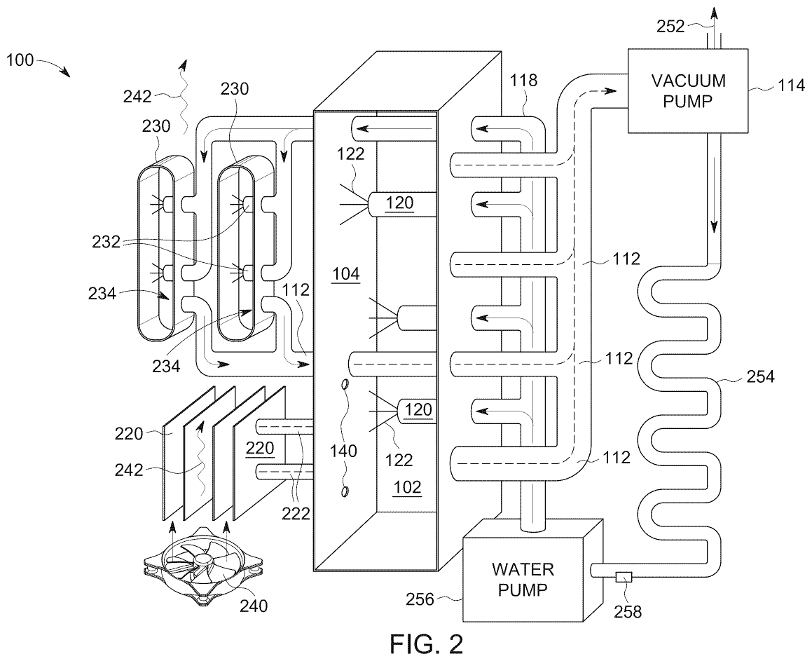

To provide a controllable method of heat removal, sensors such as thermocouples or RTDs (resistance thermometer detectors) 140 may be embedded within thermally conductive platen 104. Temperature and/or pressure sensors in the region or object to be cooled, in cooling fins (220 of FIG. 2), pressure sensors in the water circuit (such as 258 of FIG. 2), flow sensors, and the like may provide data. Data from these sensors may be monitored in real time by computer control 490 such as a computerized analysis system. By measuring the temperatures of platen 104 during the vaporization process, it is possible to determine the heat flux leaving platen 104 and ensuring the desired temperatures of platen 104 are achieved and maintained during the vacuum-driven evaporative cooling process. With appropriate sensors, electronics, and control systems, it is possible to control water flow rates, ejection speed and flow rate of water droplets, flow rates from sprayers 120, cycling the sprayers on and off, and vacuum level, thereby to control temperature, temperature reduction of the platen, rate of cooling, and time. Computer control 490 may optimize cooling against water usage, power consumption, cooling per unit of water consumed. Computer control 490 may take into account various confounding factors such as the initial temperature of the region to be cooled and materials within the region to be cooled. Control algorithms may accept input from the sensors, and use them as feedback, for example for PID (proportional-integral-derivative) controllers to optimize these parameters. These data (both the sensor data and the computed drive outputs) may be used to control opening and closing of valves such as three-way valve 132, valves to and control levels of vacuum pump 114, and valves to and drive levels of water pump 256, to thereby control spray repetition rate to maximize the cooling process per unit time and per unit volume of water or volume to be cooled/refrigerated.

In one example implementation:
Mister 120 sprays water on to the back of conductive platen 104.
   The heat of vaporization of H₂O is 2,256 joules per gram
Platen 104 may be formed of aluminum-aluminum has high thermal conductivity, but lower cost than, for example, silver.
   Dimensions: 10 cm×5 cm×0.5 cm
   Mass: 67.5 grams
   Specific heat 0.90 joules/gram/° C.

With vacuum applied to the box, the water vaporizes and draws heat from thermally-conductive platen 104.

To maintain the temperature of platen 104 at the selected temperature, additional small amounts of water may be sprayed and evacuated during the application period. The precise control of the misting and evacuation process may be determined by systemic unit testing and control algorithms included in the device based on the systemic unit testing that used the inputs from monitoring of sensors 140.

Any air remaining inside the vacuum volume may be managed, for example to flow across the surface of platen 104 to enhance evaporation or sublimation. A fan may agitate this air, or vacuum draw 112 (and therefore exhaust of the water vapor) may be arranged at one side of the vacuum chamber and the inlet at the other, to provide relatively rapid changeover of the air volume, so that evaporation may be improved.

In some cases, the water vapor may be exhausted to the environment. In other cases, the water vapor may be recaptured, condensed, and recycled in a closed system.

Flash evaporation temperature is related to pressure as follows:

| temperature ° F./° C. | pressure (mbar/atm) |
|---|---|
| 70° F./21° C. | 25 mbar/0.024 atm |
| 65° F./18.3° C. | 20.5 mbar/0.020 atm |
| 60° F./15.6° C. | 17.4 mbar/0.017 atm |
| 50° F./10° C. | 12.5 mbar/0.012 atm |
| 41° F./5° C. | 8.7 mbar/0.0086 atm |
| 32° F./0° C. | 5.7 mbar/0.0056 atm |
| 14° F./−10° C. | 2.6 mbar/0.00257 atm |
| −4° F./−20° C. | 1.0 mbar/0.00099 atm |

From room temperature to freezing, the liquid/solid phase boundary is sufficiently close to log/linear that each ° C. in temperature reduction requires a reduction in pressure of just under 2%, more or less.

Platen 104 may be coated (on either the vacuum-facing side or the environment-facing side) with a coating material, typically a chemically-inert and thermally-conductive material. Thin coatings of Teflon, nylon, or some other plastic or resin, or some other non-metallic material, may be used. The coating may reduce adhesion and tissue damage during the cooling process. The coating may protect platen 104 if it is formed of a chemically-reactive material like aluminum.

The interior, vacuum-facing side of platen 104 may have fins, a highly-cavitated surface, or other surface features to increase surface area and evaporation rate.

In some cases, misted liquid 122, such as water, may have droplet diameters ranging from approximately 200 microns to 600 microns in diameter and as a result, the surface tension of the droplets will be sufficiently high to adhere to platen 104 at any orientation. In such cases, spray device 120 and platen 104 (and thus entire apparatus 100) may be oriented at any angle.

Components of the vacuum chamber may be sealed against each other by one or more O-rings 118. The material of the O-ring may be selected to provide low volatility into the vacuum, to seal well, and to provide good insulation between cooling platen 104 and vacuum bell 110. Good materials include various synthetic rubbers, such as Viton, a brand of high density FKM vinylidene fluoride fluoroelastomer material, from The Chemours Company.

Mister 120 may be a commercial mister, or a fuel-injection nozzle, or other spray device that emits finely-divided droplets and whose flow rate is easily and precisely controlled. Because evaporation rates are closely correlated to surface area of the droplets, finely divided droplets tend to be desirable.

The vacuum may be drawn by a commercial vacuum pump 114, available from companies such as Micropump, Inc. in Vancouver Washington, which in turn is part of IDEX Corp.

Microprocessor controller 490 may be used to control various system parameters, principally (but not exclusively) water spray rate and vacuum pressure. The system parameters may be controlled moment-to-moment to:
maintain the surface temperature of platen 104 at the desired target temperature, as measured by temperature sensors 140
identify droplet icing, freezing, or fouling of the vacuum chamber, and reduce water mist rate until it's cleared or raise an alarm for the need for cleaning
   Control may be applied to water mist flow rate, power to vacuum pump 114, opening of any pressure valves in the system, etc. Process control algorithms such as PID (proportional-integral-derivative controller) may be used to balance system parameters with perturbations in the environmental factors.

### II.A. Vacuum Bell Sealing Against the Skin

Referring to FIG. 1C, in some cases, it may be useful to configure the vacuum chamber as an open-sided bell, with the object to be cooled providing the remaining side. This may be especially desirable when the cooling is to be applied to a part of the body. This is discussed in section IV.B, below.

### II.B. Electrolyte Solutions as Coolant

In some cases, the evaporative liquid may be water, either purified or straight from the tap.

The use of saline may allow a lower freezing temperature to be obtained. An electrolyte such as sodium chloride or calcium chloride depresses freezing point, varying by concentration. The solute and concentration may be chosen to select a desired freezing point for the solution. The freezing point of water falls from 0° C. at 0% sodium chloride solution, to −12° C. at 15% (by mass) NaCl solution, to −17° C. at 20% solution, and maxes out at about −20° C. at 22% solution. −10° C. is a common temperature used to impact adipose cells in the body, reachable by a 13% (by mass) solution of NaCl. −18° C., a common temperature used for commercial freezer applications, is reachable with a NaCl solution of approximately 21% by mass. Calcium chloride solution may also be used. Calcium chloride has a lower freezing point than achievable with a sodium chloride solution. A 20% solution of CaCl freezes at −18° C., and a 30% solution of CaCl freezes at about −46° C.

Referring again to FIGS. 1A and 1B, if a saline solution is used, evaporation will leave behind a residue of salt on platen 104. For typical cooling cycles required to disrupt fatty tissues, less than a gram of sodium chloride or calcium chloride will be remain upon platen 104. To remove this material at the end of a cooling cycle, a set of quick connect units 150 are used to disconnect the conductive platen 104 and the non-conductive O-ring seal 118 from the vacuum bell 110. The interior surface of conductive platen 104 may then be wiped with a cloth containing water to remove the remaining sodium chloride or calcium chloride. The unit may then be simply reassembled using quick connect units 150 for the overall vacuum apparatus 100 to be ready for the next tissue cooling treatment.

### II.C. Replacement Water

System 100 may flow the water in either a closed loop, or in an open loop with vapor exhausted and makeup water fed in as necessary. For open loop systems, the intake may have filters to remove contaminants, or purity testers to ensure that makeup water is of adequate purity.

Solutes may be used to alter vaporization temperatures/pressures. Solutes may be more readily used in closed-loop systems where near 100% of the water vapor is recovered, condensed, and reused in the cooling cycle.

### III. APPLICATION AS A REFRIGERATION OR AIR CONDITIONING SYSTEM

Referring to FIG. 2, a refrigeration or air conditioning device 100 may use vacuum-driven evaporative cooling for air conditioning or refrigeration (in either case, lying at the left side of FIG. 2). A vacuum chamber 102 may be formed as an open space with spray misters 120 configured to spray 122 onto a conductive platen 104. Vacuum pump 114 may draw vacuum 112 into volume 102 facing platen 104. Thermal sensors such as thermocouples or RTDs 140 may be placed in platen 104 to monitor the heat flux and temperature of platen 104. Cooling fins 220 may be thermally connected to platen 140 by thermally conductive bars or by a convection cooling loop 222. Alternatively, multiple cooling chambers 230 may be provided, each having sprayers 232 and vacuum exhausts 234. Fins 220 or cooling chambers 230 may be either in, or in a duct for flow into, a cooled region which may in turn be an enclosed volume, such as a refrigerator or cold-chain chest for delivery of pharmaceuticals or other temperature-sensitive medical supplies or materials, or may be an open volume, such as a room to be air-conditioned.

As platen 104 or cooling chambers 230 are cooled due to the vaporization of liquid 122, a device 240, such as a fan, may force cooling air or air from the room to be cooled 242 to flow along the outer surface of platen 104, through fins 220, or past cooling chambers 230. Air flow 242 may be cooled and then directed to desired regions within the refrigeration device or to the room to be cooled.

Vacuum pump 114 may be situated exterior to the space for which cooling is desired. This allows the heat generated during operation of vacuum pump 114 to be dissipated into the ambient environment, without radiating back into the region where cooling is desired. The vapor generated from the vaporization of the liquid in the vacuum volume 102 may be exhausted 252 to the exterior of the desired region to be cooled, or may be forced through condenser 254 where the vapor is converted back to a liquid phase. The condensed liquid may then be recycled through water pump 256 to be sprayed through misters 120. This closed loop system does not release any of the coolant to the outside environment.

### III.A. Application as a Refrigeration or Air Conditioning System with Concurrent Humidification of an External Region

Referring again to FIG. 2, water vapor removed from vacuum pump 114 may be exhausted though exhaust port 252 into a region outside of the volume to be cooled. The exhausted water vapor will increase the humidity of the region into which it is exhausted. As such, the system may also provide humidification to regions while cooling other regions within a selected environment. Water may be added (118 of FIG. 1A) to the system as required to replenish water vapor exhausted into the environment. This may, in turn provide “swamp cooler” cooling for that exhausted-into region. For regions of the globe with very low humidity, this combined cooling at surfaces 104, 220, 230, plus humidification 252 and lower-level cooling by humidification may enhance the characteristics of habitable spaces in those regions.

### III.B. Prevention of Water Freezing

Sensor 258 may measure pressure and/or flow of water or vapor at any point in any of the flow paths, such as at the intake to water pump 256 from vacuum pump 114 and condenser 254. In a closed-loop system, the rate of return of water from condenser 254 or from vacuum pump 114 per unit time should equal the output of water sprayers 120, 232. If sensor 258 detects that flow into water pump 256 is lower than the flow out from pump 256 to sprayers 120, 232 then it may be inferred that there is an ice buildup, either on platen 104 or on sprayer 120, 232. Buildup of ice on platen 104 may also be observed via an optical sensor, such as a high-resolution camera placed to have a view of the facia of the platen 104 with an LED on the opposite side of the chamber, so that glare will be easy to see. Alternatively, icing may be detected via an illumination level detector placed in the platen below the points where icing is most expected. Ice buildup may adversely impact the cooling process, either by acting as an insulator or by otherwise impairing evaporation of droplets 122. If computer control 490 determines that such an event has occurred, then the system may automatically alter the valving/vacuum pumping process, for example by allowing entry of warm air, by reducing water flow, by drawing down the vacuum level to increase sublimation, by increasing the speed of fan 240 to blow more heated air over cooling fins 220, or by shutting off fan 240. When computer control 490 has determined that the ice buildup has been mitigated (using, e.g., temperature sensors 140 or flow sensor 258), then the original cooling process will be reinstated to cool the refrigeration region.

### III.C. Water Droplet Control

Cooling system 100 may use a specially designed nozzle for water sprayers 120. Materials may be chosen to minimize corrosion, oxidation, and erosion via pressurized flow. The nozzle may be shaped to provide droplets with a desired size, and to some degree, shape in the first instant, before surface tension draws the droplet into a spheroid. Stainless steel and similar materials may be desirable, and thus the material should be chosen to permit precise shaping. Efficiency of vacuum-driven vaporization may be higher with higher surface area to volume ratios of the water droplets 122, which tends to favor more-fractionated, smaller droplets. However, droplets 122 should have sufficient mass to be accelerated to reach platen 104, rather than vaporizing entirely before reaching platen 104. Smaller droplets may permit lower amounts of water to be required in the system for the same cooling outputs. For under-developed regions of the world this aspect may provide significant benefit.

Optimization of the direction and ejection angle of water sprayers 120 may enhance the efficiency and capability of the overall cooling system 100. In particular, by appropriate design and manufacture of the nozzle for the sprayer, the direction, ejection angle, and dispersion angle from the nozzle may be chosen to cover platen 104 with a uniform surface of water droplets. This approach will maximize the heat transfer to the platen 104 upon vaporization of the water droplets 122 when the vacuum is applied to the chamber 102. Thus, the cooling of the regions to refrigerated/cooled will be optimized and minimize the time to achieve the desired temperature of the cooled region.

### IV. COOLING FOR MEDICAL AND THERAPEUTIC APPLICATIONS

### IV.A. Cryolipolysis

Referring again to FIGS. 1A, 1B, and 1C, cryolipolysis is a method for removing adipose tissue by cooling. The method involves controlled application of cooling within the temperature range of −11° C. to +5° C. Subcutaneous fat tissue is selectively sensitive to temperatures in this range. While the process is not fully understood; it appears that fatty tissue that is cooled below body temperature, but above the temperature at which tissue freezes, undergoes localized cell death (“apoptosis”), or the cells dissociate from the tissue matrix, followed by a local inflammatory response that gradually over the course of weeks to months results in elimination of the fat cells from the body, and thus a reduction of the fatty tissue layer. Cooling into this range tends to leave other cells, such as skin and nerve cells, undamaged. For example, overlying skin tolerates exposures to −10° C. for periods of a half hour to an hour without apparent damage. Cryolipolysis may be used as a noninvasive, localized reduction of fat deposits, reduce lipid-rich cells and fatty tissue, to reshape the contours of the body, for cosmetic or therapeutic reasons.

Vacuum-driven evaporative cooling apparatus 100 may provide desired cooling of tissues of the body in a rapid and controlled manner. Highly thermally conductive platen 104 coated with a thin layer of a non-metallic material may be placed in contact with desired region of tissue 98. The thin, nonconductive coating may prevent conductive platen 104 from adhering to tissue 98 when the temperature is lowered below 0° C., for example, because of freezing of water at the surface of the skin.

Computer control 490 may read temperature sensors 140 and adjust water flow rate and vacuum pressure to control cooling to maintain a desired temperature and rate of cooling, to correct for various confounders such as variations in blood circulation that results in variations in supply of heat back into the tissue. Lower temperatures may be achieved by either lowering the vacuum pressure or adding electrolyte to the water being injected. Increased rate of cooling to a fixed destination temperature may be achieved by faster insertion of water and withdrawal of water vapor.

As an example of an application of this cooling process, the tissue may be cooled into the range where fat cells are selectively disrupted, and other tissues are not injured. In order to avoid frostbite, a specific temperature level and exposure may be determined, such as 45 minutes at −10° C. (14° F.), that injures the fat but not surrounding tissues. The system may be driven to apply the desired degree of cooling, to a layer of fat below the skin, typically 1 cm or a little more, per treatment.

The thermally-conductive platen may be flexible or conformal to allow platen 104 to conform to various body parts. A conformal platen may be constructed of multiple thin sheets of aluminum, each sheet polished smooth to allow the sheets to slip against each other with minimal lubricant so that the platen as a whole offers thermal conductivity approximating that of solid aluminum, but the whole stack sufficiently rigid to support vacuum.

In one example implementation:
Mister 120 sprays water on to the back of conductive platen 104.
Platen 104 may be formed of aluminum-aluminum has high thermal conductivity, but lower cost than, for example, silver.
   Dimensions: 10 cm×5 cm×0.5 cm
   Mass: 67.5 grams
   Specific heat 0.90 joules/gram/° C.
Thin, non-conductive material may be a Teflon liner
   Dimensions: 10 cm×5 cm×0.05 cm
   Mass: 5.5 grams
   Specific heat of Teflon: ˜1.5 joules/gram/° C.
Tissue 98
   Dimensions: 10 cm×5 cm×1 cm
   Mass: 45 grams

Specific heat of tissue: 3.47 joules/gram/° C.

If tissue, Teflon and thermally-conductive platen begins at 37° C., the combined system drops ˜10.0° C. per gram of water applied to platen 104. So, to have the tissue surface reach a target temperature −10° C., approximately 5 grams of water will need to be misted onto platen 104. This tissue surface temperature has been used in previous efforts with thermoelectric cooling systems to enable disruption of fatty tissue without damage to the skin surface of the tissue 98.

To account for blood flow heating, additional small amounts of water would be sprayed and evacuated during the application time period. The precise control of the misting and evacuation process would be determined by systemic unit testing and control algorithms included in the medical device based on the systemic unit testing that used the inputs from monitoring of sensors 140.

### IV.B. Vacuum Bell Sealing Against the Skin

Referring again to FIG. 1C, a vacuum bell 110 may be placed in contact with desired region of tissue 98, without the intervening platen. Vacuum bell 110 may be sealed against tissue 98 via an O-ring, petroleum jelly, or similar sealant. Water spray mister 120 may provide a mist of water 122 directly to the outer surface of tissue 98 within the volume of the vacuum bell 110, and vacuum pump 114 may draw vacuum 112 in volume 102 between vacuum bell 110 and tissue 98. If there is no platen 104, thermal sensors such as thermocouples or RTDs 140 may be placed on tissue surface 98. This approach may provide more rapid cooling, and is suitable where skin 98 is sufficiently thick and robust to tolerate applied vacuum and cooling without injury (for example, hemorrhaging or excessive evaporation). Where the skin or other tissue is less tolerant to vacuum, the platen approach of FIGS. 1A and 1B may be desirable.

### IV.C. Cooling for Analgesia

As another example, the cooling may be used to provide analgesic effects to selected regions of the body.

Cooling tends to reduce the perception of pain. Cold therapy causes decreased nerve conduction velocity and other local effects to lessen the sense of pain perceived by peripheral nerves in the skin. Another conjectured mechanism of action is hypothesized: at the point where cold-sensing peripheral nerves reach the spinal cord, activation of cold-sensing receptors may interfere with pain-sensing nerves, reducing the perception of pain. Cooling one part of the body is known to reduce the perception of pain from elsewhere in the body. The effect seems to be larger for chronic pain such as arthritis, phantom-limb pain, or neuropathic pain. Cooling is also effective for pain of burns.

The vacuum-driven evaporative cooling device may be used to reduce pain by cooling specific portions of the body to specific temperatures, that vary with the part of the body and nature of the pain. Computer control 490 may be programmed to apply that level of liquid, e.g., water, and vacuum appropriate to apply the appropriate cooling for the patient's pain.

### IV.D. Cooling for Hypopigmentation

As another example the cooling may be used to provide skin lightening to selected regions of the body.

Hypopigmentation has been observed as a side effect of temporary cooling or freezing of tissue. Loss of skin pigmentation may occur due decreased melanin production, decreased melanosome production, destruction of melanocytes, or inhibited transfer of melanosome into the keratinocytes in the lower region of the epidermal layer. While some hypopigmentation devices and systems have been developed, it may be desirable to effect improvements in this area. The methods and applications described herein may improve the consistency of the skin cooling or freezing and may improve the consistency of the duration of the skin freezing in a non-invasive manner. Such improvements may be desirable to improve overall hypopigmentation consistency.

### IV.E. Cooling for Pharmaceutical Transportation Cold Chain

During transport of pharmaceuticals, blood products, organs for transplantation, or other temperature-sensitive medical supplies or materials, electrical power from a traditional stationary electrical source, such as an electrical outlet connected to the electrical grid, may be unavailable or inconvenient. Electricity for a cold transport chest may be provided by a portable power source, for example, solar cells that convert sunlight into electricity. A small transport cooling chamber that utilizes vacuum-driven vaporization is expected to consume approximately 40 to 60 watts. Sufficient electrical power may be provided by approximately 2,500 square centimeters of solar cells (5 kW/square meter/day as the solar flux). This array of solar cells may be configured as a square array of 50 cm on a side. Since this area is larger than a typical pharmaceutical transport container, foldable solar cells may be used. This may allow the solar cells to be folded together for ease of initial conveyance and then unfolded to acquire solar energy as needed during pharmaceutical transport. A switchable connection may allow the cooling system to be alternatively switched between the solar array and a traditional fixed-location power outlet.

### IV.F. Cooling for Tissue Ablation within the Body

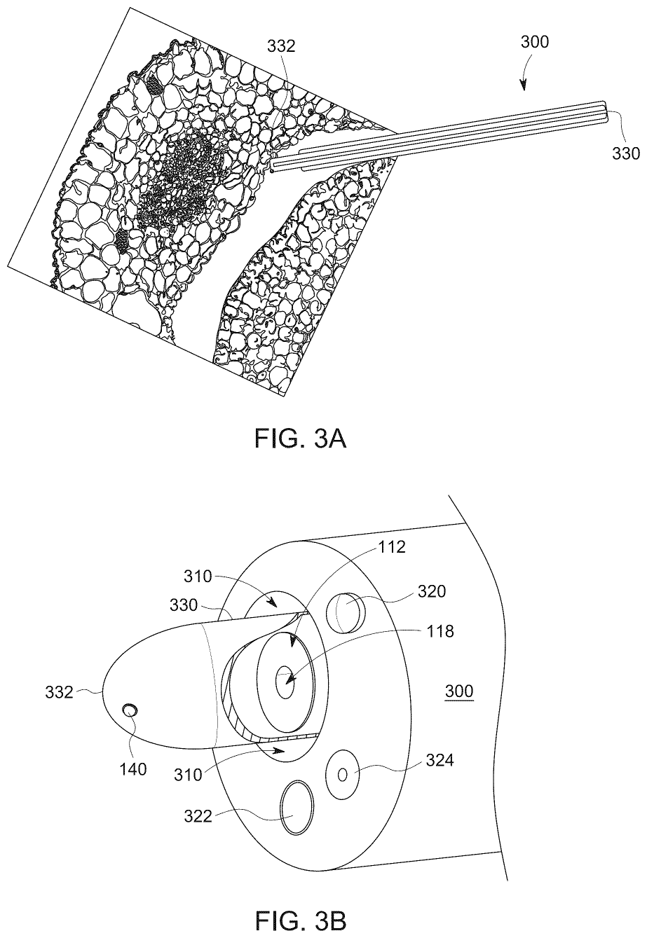

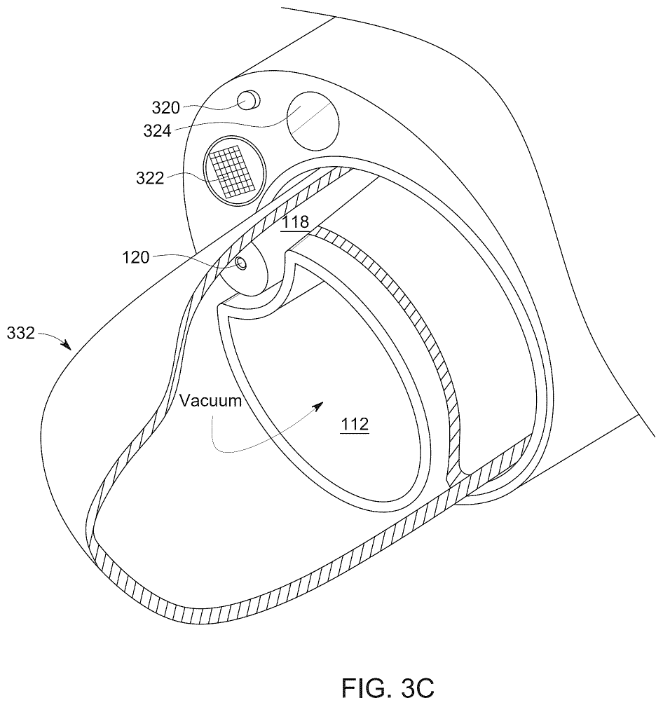

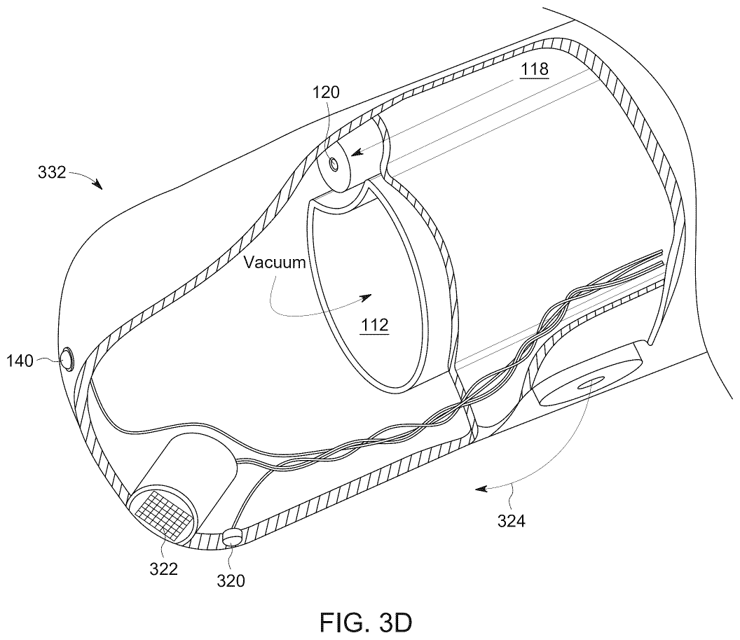

Referring to FIG. 3A, tissue within the body may be cooled to ablate undesired cells from the tissue linings. Endoscope 300 may be inserted into the body through a natural orifice. For example, a bronchoscope may be advanced through the trachea to the selected generation of the lung, i.e., trachea, main bronchi, lobular bronchi, or segmental bronchi. Or a gastroscope may be advanced through the mouth to access the esophagus, stomach, duodenum, or small intestine. Referring to FIG. 3B, endoscope 300 may be selected to have the largest available working channel 310, e.g., for a 6 mm outer diameter bronchoscope with a 2 mm working channel to pass instruments through the bronchoscope or for the gastrointestinal tract, a 10 mm outer diameter endoscope with up to a 2.8 mm working channel to pass instruments through the scope.

Endoscope 300 may have an illumination source 320 and objective lens or CCD camera 322 that may allow the operator to visualize the passages within the body. Endoscope 300 may have air/water nozzle and/or water jet 324 features that permit the operator to clear undesired materials from the path of endoscope 300 to enhance navigation to the desired location within the body. When endoscope 300 is at the selected position within the body, a catheter 330 with multiple lumens may be passed through the working channel of the endoscope with the catheter tip extended a short distance beyond the tip of the endoscope. Catheter tip 332 may be a solid unit or an expandable member. It may be a conformal, highly thermal conductive material coated with a thin layer of a non-metallic material. Catheter tip 332 may then be placed in contact with the wall of the tissue at the selected position within the body. A specified amount, say 1 gram, of a liquid, e.g., water, is then injected into the catheter inner water-feed lumen 118, the liquid transport tube, by a suitable device (not shown) from outside of the body.

The liquid is advanced to the tip of the catheter where it accumulates in the outer vacuum-draw lumen 112. A vacuum is then applied to outer vacuum-draw lumen 112 of catheter 330 by the use of vacuum pump 114. When an appropriate vacuum level is attained (roughly less than 10 Torr), the liquid will vaporize and tip 332 of the catheter outer lumen 112 (and the temperature of the tissue with which it is in contact) will cool significantly due to the absorption of the heat of vaporization from the tissue. A thermal sensor 140, e.g., a thermistor or thermocouple, is placed at tip 332 of outer vacuum-draw lumen 112 to measure the temperature of the impacted tissue to ensure the desired temperature is achieved by cooling.

By the appropriate selection of the volume of the liquid that is vaporized and the composition of the liquid (for example, water, sodium chloride solution, or calcium chloride solution), the tissue may be cooled to below −20° C. and within a controlled depth of the tissue (e.g., 1 to 5 mm depth). This may enable the ablation of undesired cells at the selected location, such as excessive goblet cells found with chronic bronchitis. Due to the nature of the cryobiology, the epithelium returns to normal epithelium following ablation, e.g., with the vast majority of the goblet cells eliminated from the bronchial tissue.

When the catheter is to be moved to the next location of the tissue to be cooled, a warm liquid or warm air may be passed through lumens 118, 112 the catheter to increase the temperature to a level where no damage to the tissue may be caused due to the catheter tip “sticking” to the tissue by freezing.

A sequence of cooling of the desired linings of tissues may enable normal epithelial tissue linings to return to the selected regions. This approach may be used, for example, in the airways to ablate segments of the segmental, lobular, main bronchi and the trachea may be employed to ablate the undesired cells, such as excessive goblet cells, from the selected bronchi with a return to normal epithelium in each location.

Referring to FIGS. 3C and 3D, both functions may be combined into a single catheter that provides vacuum-driven evaporative cooling, mechanical delivery, and endoscopic optical visibility to guide the cooling to the precise location at which treatment is to be provided. The tip of the catheter may be formed primarily of an aluminum or steel globe that supports vacuum. Vacuum may be drawn through a lumen acting as a vacuum-draw channel 112. A sprayer or mister 120 may spray water or a saline solution into the vacuum globe. A thermocouple or other sensor may be embedded in the wall of the vacuum globe to measure temperature of the globe at the treatment site. Camera 322 may be mounted with a lens projecting through the vacuum globe, preferably at the side of the globe away from where water is sprayed 120. The catheter may have a smooth outer surface so that the catheter can easily be rotated, to alternate between camera view and then a touch of the cooling surface of the vacuum globe.

Conditions that may be treated include diseases of the esophagus such as esophageal cancer or Barrett's esophagus, diseases of the respiratory tract, diseases of the stomach or intestine or rectum.

### V. ADDITIONAL SPRAY IMPROVEMENTS

Using vacuum driven evaporation of water to provide cooling and refrigeration avoids the use of high global-warming potential refrigerants that can adversely impact the environment and drive climate change.

### V.A. Apparatus

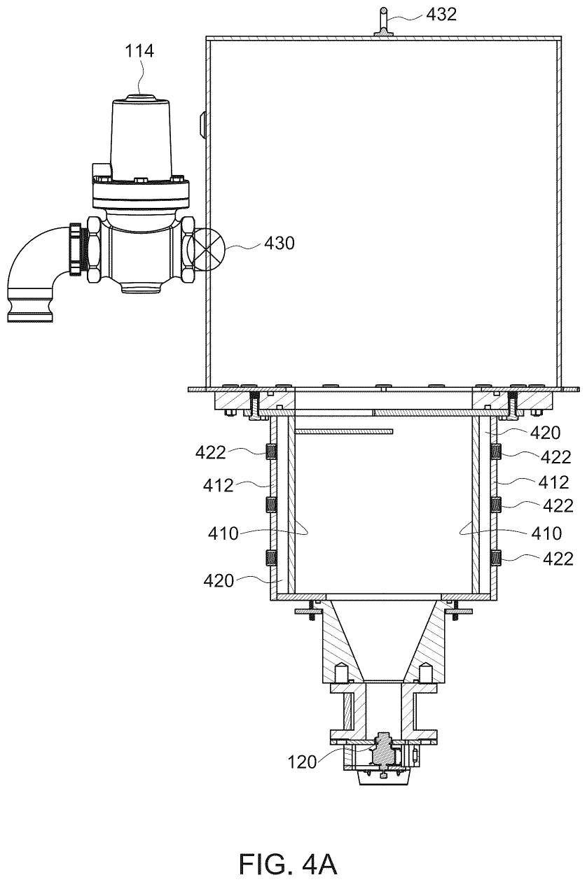

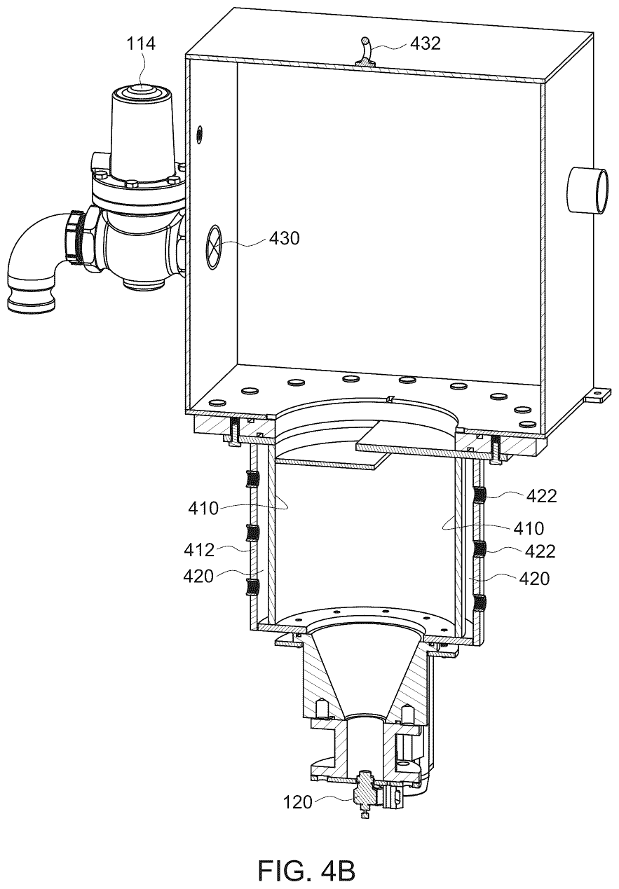

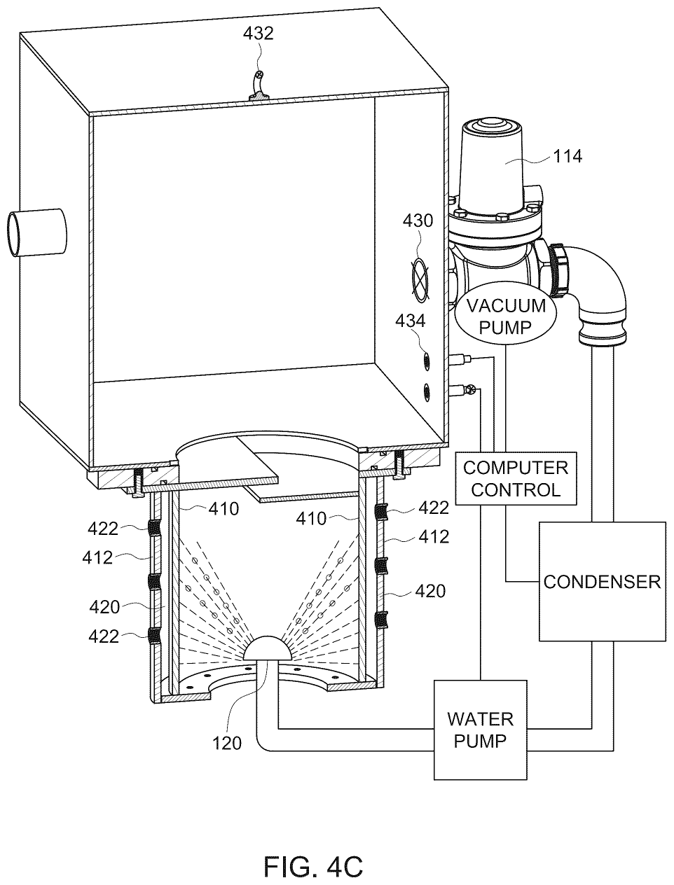

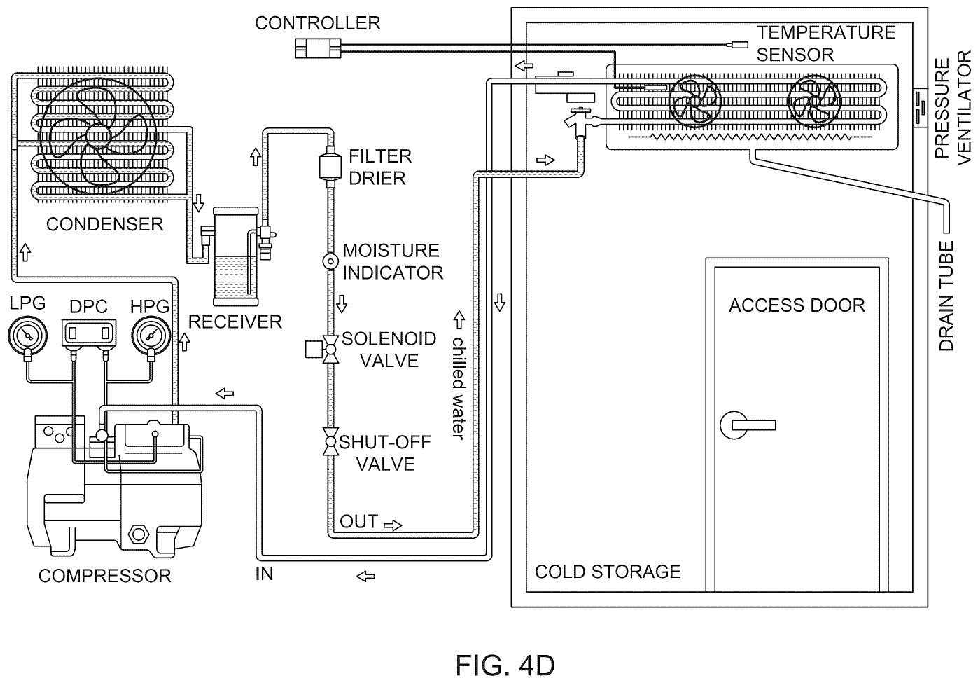

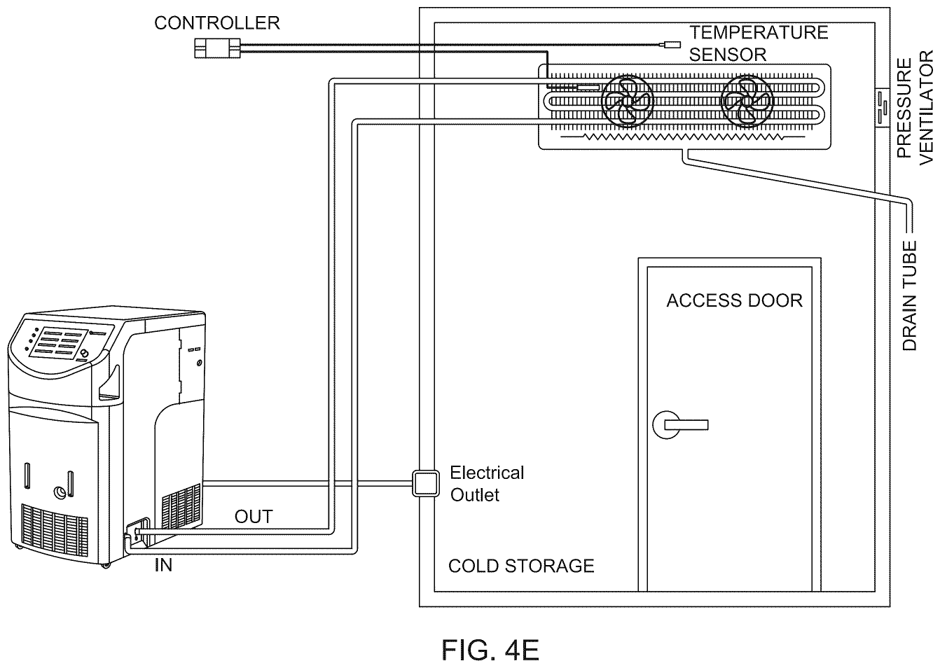

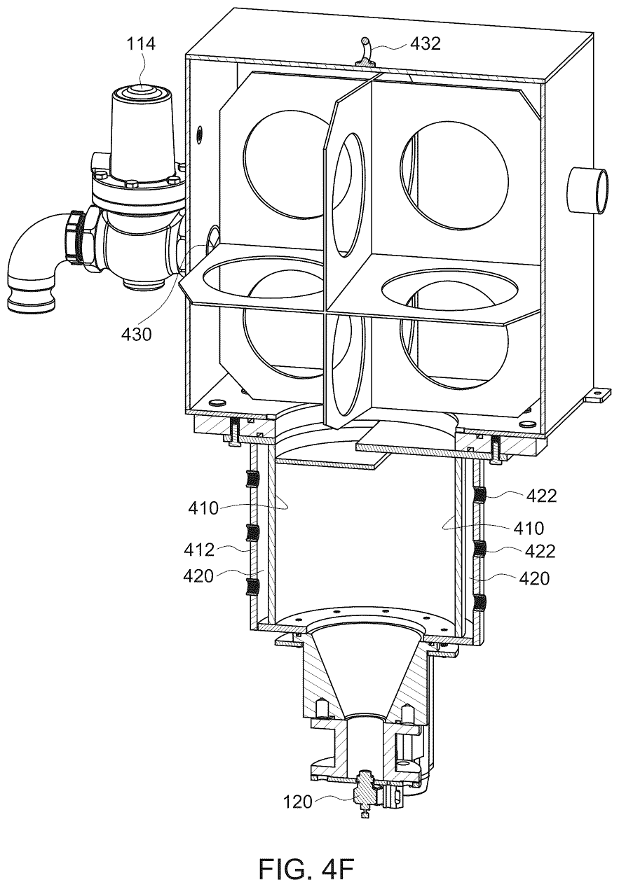

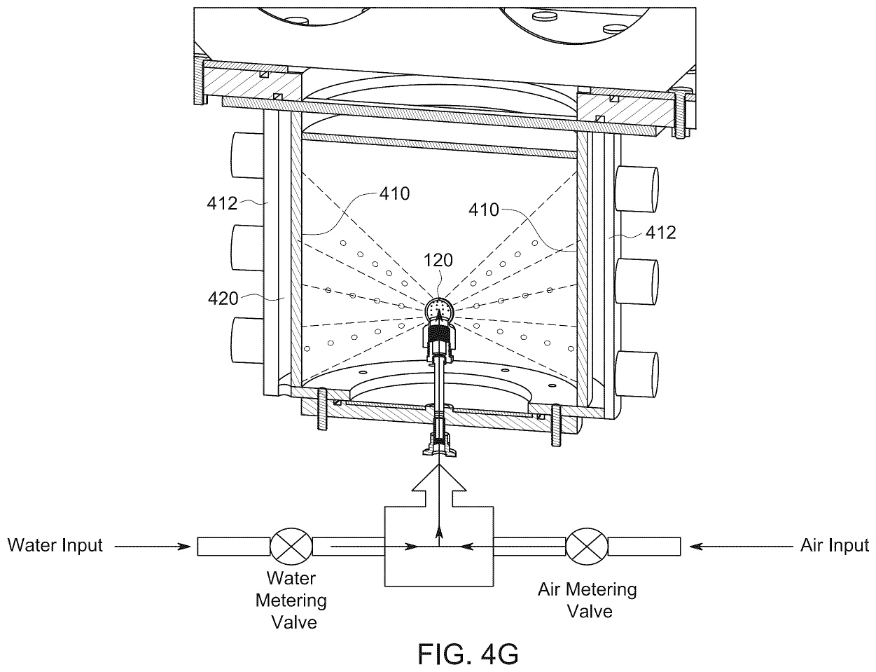

Referring to FIG. 4A, atomizer 120 may be installed at the bottom of conical antechamber 408, which leads to a cylindrical evaporation chamber 410, which may have an adjacent upper chamber 450. Sprayer 120 may be selected to spray onto the walls of inner aluminum cylinder 410. The conical antechamber may be arranged to have an angle corresponding to (and no smaller than) the dispersal spray cone from atomizer 120, so that most of the droplets will hit the wall of cylinder 410. A cylinder is efficient because it corresponds to the dispersal pattern of water droplets from atomizer 120, and it has no corners that are easily missed by the spray cone and that create vortices and dead spots for the circulation of water in water jacket 420. The dimensions and location of the cylinder wall may be tailored to the dispersion pattern of sprayer 120, and vice-versa. For example, if the sprayer sprays a pattern from 45° elevation to 0° elevation, cylinder 410 may be arranged to subtend that portion of the arc around sprayer 120. If sprayer 120 has a pattern from 50° to 70°, conical antechamber 408 may have depth and diameter to position cylinder 410 in that dispersal pattern.

Atomizer 120 may be chosen to mix air into the spray to yield droplets of 20μ to 50μ in diameter. Alternatively, sprayer 120 may be driven by water pressure only, with no air, and may spray droplets of 80-100μ. Droplets this small will stick to inner surface 410 of the cylindrical evaporation chamber by surface tension. Larger droplets may be absorb more heat, but at the tradeoff of less controlled adhesion to cooling surface 410 of the cylindrical evaporation chamber. Cooling surface 410 may be roughened or given some other surface treatment to improve stickiness to water surface tension.

An inner cylinder 410 and outer cylinder 412 may form water jacket 420 that functions as a heat exchange and a convective heat transport to the cold room where cooling is desired. Inner cylinder 410 may be aluminum because of its high heat conductivity from the water evaporating on its inner surface to surrounding water jacket 420. Water in water jacket 420 may be introduced by multiple input ports 422 and removed at multiple output ports 422, for example three of each, to prevent dead spots in water jacket 420, which improves heat transfer. The cylinder may be about 10″ diameter and 10″ tall, for a total volume of about 7 liters. Upper chamber 450 may be a cube about 20″ on a side. The passage between the cylindrical evaporation chamber and upper chamber 450 may be covered by baffles that overlap in the center. Two or more baffles may each cover slightly more than half the cross-sectional connection between upper chamber 450 and cylindrical evaporation chamber 410, so that gas may flow freely between, but baffles between prevent the spray from atomizer 120 from reaching upper chamber 450. The cube dimensions may be chosen to provide some volume of air. There is a temperature- and pressure-varying limit of how much water vapor can be dissolved in a given volume of air; that limit forms the dew point. To increase the amount of water vapor that can be pumped out per unit of time requires the requisite amount of air that can dissolve that much water vapor. The size of upper chamber 450 may be optimized relative to the pump's pumping rate to provide a large enough volume of air to dissolve the necessary amount of water vapor, but small enough that vacuum pump 114 can pump it out efficiently. Vacuum pump 114 may be designed to only pump air and water vapor, and only to rough vacuum, and need not be further designed to handle flammable, explosive, toxic or corrosive gasses, which may allow optimization of vacuum pumping speed.

At the upper left of FIG. 4A (upper right of FIG. 4C) is the exhaust tube and vacuum pump 114. Valve 430 can close or open the exhaust. At the upper right of FIG. 4A (upper left of FIG. 4C) is vent valve 432. At the middle right of FIG. 4C is thermocouple vacuum gauge 434. Commercial thermocouple vacuum gauges have an effective range from 1 atm to 10-3 mbar.

The apparatus may use a dry screw pump 114, for example from Leybold, that is specifically designed to operate with vapors. A Leybold ND65 is able to pump 1.9 liters of water vapor per hour. Alternatively a Leybold ND200 pump may be used. The pump may be chosen to have a capacity that meets the total amount of cooling needed for a given refrigeration, freezer, or air conditioning system. For commercial systems, a higher water vapor capacity pump may be used these pumps are available in the commercial vacuum pump market. A refrigeration system may optimize the pump choice and design for maximum water vapor capacity with high pumping speeds.

Referring to FIGS. 4D and 4E, a water-based cold/refrigeration cycle may be substituted into an existing cold cycle, by substituting the vacuum-driven water-evaporation-based cooling chamber for a Freon-based or other high Global Warming Potential refrigerant chiller.

Referring to FIG. 4F, upper chamber 450 may be braced by an insert that braces against vacuum pressure. This brace may reduce problems with upper chamber 450, for example metal stress and fatigue due to repeated vent/vacuum cycles of the system.

Referring to FIG. 4G, sprayer 120 may have a full spherical distribution, or with a distribution that is most of a full sphere (for example, 240° coverage to spray against the inner walls 410 of aluminum cylinder, with or without spray coverage against the top wall) may be placed in the center of a cylindrical heat exchanger and water jacket 420.

The spray may be an atomizer 120, where the spray combines water and gas (air, nitrogen, etc.) with the expansion of the gas contributing to breaking up the droplets to be a small mist, or may be a sprayer 120 of water only, where the formation of droplets is effected by the water passing through the spray orifice. For a water/gas atomizer 120, it may be important to ensure a pressure balance between the water flow and gas flow into atomizer 120, or to provide anti-backflow valves, to keep water from flowing up the gas feed line, or vice-versa.

### V.B. Operation

### V.B.1. A First Cycle

The cycle may begin with the vent valve closed, the vacuum valve closed, the sprayer valve closed, and vacuum pump 114 in operating mode. The chamber may be at atmospheric pressure or below.

The air vent valve may be opened to bring the volume to atmospheric pressure or to some pressure relatively near atmospheric. The sprayer valve may then be opened. A spray of water droplets from atomizer 120 impacts on the sides of aluminum cylinder 410. The spray may include air which atomizes the spray into droplets. Following a pre-determined period of time sprayer 120 is shut off, the atomizer valve is closed and the vent valve is closed. The amount of water may be regulated by time, or by volume. For example, the water spray may be 5 seconds or 10 ml of water.

The vacuum valve may be opened and vacuum pump 114 may run for a time (typically about 30 seconds) to lower the pressure in the vacuum chamber. This drives vaporization at lower temperatures. As the pressure in the chamber is lowered, the temperature at which the water droplets will vaporize also lowers. For example, at 7 mbar of pressure the water evaporates at 2° C. This enables the aluminum cylinder to be significantly cooled (2,260 Joules per gram of water evaporated), and that cold is carried away by the water circulating through water jacket 420. This cooling may then be used in commercial refrigeration, cold rooms, air conditioning, etc.

Running vacuum pump 114 and opening or closing the valves may be controlled based on pressure within the chamber. After atomizer 120 has sprayed water and the vent valve and atomizer valve closes, then the vacuum valve is opened. The vacuum gauge may monitor the precise pressure within the vacuum chamber. As the pressure drops due to vacuum pump 114, the thermocouple vacuum gauge may monitor pressure. When the last amount of water evaporates, the rate of pressure change will suddenly accelerate. When that rapid pressure change is detected, or pressure reaches below 3 mbar (for a refrigeration system), computer control 490 may close the vacuum valve. Computer control 490 may shut off vacuum pump 114.

Next, the vent valve will open to bring the chamber back up, perhaps to atmospheric pressure, perhaps to some lower vacuum, perhaps to a pressure just above. When the vacuum sensor detects that pressure has reached the restart point, the cycle will repeat.

A control algorithm based on pressure may give improved results relative to a control based on timing. For example, the water flow rate and flow through atomizer 120 may be somewhat imprecise, for example ±10%. A pressure-based control may self-compensate for this variability.

### V.B.2. A Second Cycle that Reduces Cycling from Vacuum to Ambient Pressure

An operating cycle may be designed to reduce cycling from the pressure at which cooling at the lowest temperature occurs, back to room ambient pressure, and back to vacuum. In this second cycle, vacuum may be maintained in the cylinder and chamber relatively near the pressure that achieves the desired cooling temperature. The pump may be cycled on and off to maintain the pressure near the pressure for the desired evaporation temperature. The cycling may be controlled by a temperature sensor or a pressure sensor.
Step 1: the chamber begins at low pressure, for example, about 5 to 7 mbar (0.0049 to 0.0069 atm or 500-700 Pa). The pressure may be chosen to achieve a desired cooling temperature. For example, water vaporizes at approximately 2° C. under a pressure of 5.3 mbar (0.53 kPa).
Step 2: In some cases, it may be desirable to open the valve of the air inlet, and allow air or other gas to flow in, to raise pressure to about 30-50 mbar (3000 to 5000 Pa or 0.030 to 0.049 atm). The presence of some gas may improve the efficiency of vacuum pump 114.
Step 3: open the water valve to allow water to flow through sprayer 120 to land on the inner walls of cylinder 410. In one embodiment with a cylinder-plus-chamber volume of about 132 liters, the water injection may be about 15-20 ml of water. Since the chamber is under vacuum, atmospheric pressure (approx 1000 mbar) may drive water into the water sprayer 120. The quantity of water may be chosen to be large enough to maximize evaporation and heat flow rate, and small enough to avoid either freezing or failing to evaporate. The injection of water may be controlled either by time (about 0.7 seconds) or by pressure. Evaporation of this water may raise pressure to about 100 to 300 mbar (10 kPa to 30 kPa or 0.1 to 0.3 atm), which a typical pump can pump down in a few seconds.
Step 4: In some cases, more air may be allowed to flow in through sprayer 120, to target a pressure range where vacuum pump 114 is most efficient. For example, some pumps are most efficient in the range of 50-100 mbar (5-10 kPa). This air will ensure that water is cleared from sprayer 120 so when pressure is reduced, there will be no ice formation on the outlet of sprayer 120. Also, the pump may be more effective in pumping a mixture of water vapor and air than water vapor alone.
Alternatively, vacuum pump 114 may be allowed to run until the pressure is slightly below the pressure for the desired evaporation temperature. For example, if the target temperature is 2° C., then the valves may be held shut and vacuum pump 114 allowed to run until the pressure is just below the corresponding pressure of 5.3 mbar (0.53 kPa). Then computer control 490 may shut off vacuum pump 114. In a few seconds, enough water will evaporate to raise the pressure just above 5.3 mbar. Then computer control 490 may turn vacuum pump 114 back on.
Return to step 1. pumping down to the pressure at which vaporization occurs at the desired temperature to effect evaporation of the water, and cooling of the cylinder wall.

The water inflow at step 3 is limited by the capacity of vacuum pump 114 to pump out the water vapor. Likewise, water flow may be reduced or limited when the desired temperature for the target space is reached. Generally, water volume should be as high as possible within those two constraints, and then the pump should be cycled off.

The water vaporization that generates the coldest temperatures is at the lowest pressure attainable in the vacuum system. So, for example, if the pump achieves a 7 mbar vacuum, then vaporization within the cylinder will occur at 2° C. (that is not necessarily the temperature achieved, only the temperature at which—or any higher—vaporization occurs). The challenge at such a low pressure is to pump as much mass of water in light of the constraints of maximum vapor content that may be attained in an air/water vapor environment. A pressure cycle from a low of about 7 mbar to a high of about 100 mbar may be used. The mass flow at 100 mbar is approximately 20× higher than at 7 mbar so more water is vaporized at the higher pressure. With optimization of the pressures in the pump-down curve and the optimization of the amount of water that may be vaporized along that pump-down curve, this approach may achieve the desired level of cooling and reach the target temperatures in the volume to be cooled.

As long as pressure is above 5.7 mbar, the freezing temperature will remain above 0° C., and icing should not be an issue. In some cases, it may be useful to spray air though sprayer 120 for a short time, to remove any icing by blowing it off, or by using room-temperature air to melt the ice.

### V.B.3. Computer Control

The control of when to open and close the vacuum and vent valves may be done by timing. For example, the vent valve may be opened for 2 seconds to bring the vacuum chamber to atmospheric pressure and then to enable atomizer 120 to spray water droplets for say 5 seconds. These valves may then close and the pump valve may open to evacuate the chamber and drive vaporization of the sprayed water droplets. Alternatively, timing may be driven by vacuum gauge 434.

Pump rate, size of the cylinder and chamber volume, amount of water per injection cycle, and amount of air per injection cycle all interact with each other, and the interactions are non-linear. A multi-factor parameter search may be used to determine optimized control parameters.

Computer control may be optimized using factorial experiment optimization techniques. The optimization problem is to maximize the output heat transfer efficiency as a function of the system inputs. Input factors that may be varied include (but are not necessarily limited to):
Water volume to be evaporated in vacuum chamber (typically milliliters per unit time in seconds)
Vacuum-pumping speed of pump to evacuate the pumping chamber (typically in cubic meters per minute or liters per second)
Vacuum pump-down cycle time (in seconds)
System volume (in cubic meters or liters)
Cooling water jacket 420 flow rate to maximize heat transfer from aluminum cylinder to cold volume (typically in liters per minute).
   Each factor may be tested at a set of parameters, the specific values for the factor.

Since each factor interacts with the other, this is a non-linear optimization process. Factorial experiment techniques may be used to find an optimal parameter set. In a linear system, an optimization approach is to hold four factors constant and only vary the fifth to determine the highest cooling efficiency as that factor is varied. This approach will enable a rapid deduction of the optimum parameters (essentially five sets of single-parameter variations). In a system where the output follows a linear relationship to its inputs, such an optimization process may work very well. However, since each factor in this system interacts with the others and is directly dependent upon the other factors, a simple one-factor optimization will not necessarily yields the best results.

To use a factorial-optimization process, each of the five factors is quantized at a finite set of parameter levels, and the system is tested at each parameter set from the cross-product set of all the parameter quantizations. For example, a first run may test three values for each of the five factors, which yields 35-729 parameter sets. In actuality, due to the physics of the system including the relationship of vacuum pumping speed and chamber volume, a number of the parameters will be focused on a more narrow range of possible values to provide the optimum cooling efficiency.

### Example 1

Volume of chamber: 2 liters.
Vacuum pumping speed: 1.01 cubic meters per minute.

| Condition: | Average Time | | |
|---|---|---|---|
| Water | to evacuate from | | Delta T of |
| added to | atmosphere to | Delta T of | cylinder |
| chamber | 3 mbar | cylinder | (Degrees F. |
| (ml) | (seconds) | (Degrees F.) | per second) |
| 0 | 3 | 0 | 0 |
| 5 | 84 | 17.3 | 0.21 |
| 10 | 133 | 22.3 | 0.17 |
| 15 | 223 | 25.2 | 0.11 |

In this simple example where the volume of the chamber has been fixed at 2 liters and the vacuum pumping speed is fixed at 1.01 m³/min, one may observe that the most efficient cooling is found with the input of 5 ml of water (0.12° C. per second).

To move into the next phase, one would vary the pumping speed and repeat the measurements to find the optimum pumping speed for this chamber volume to achieve the highest level of heat transfer. Then once an initial estimate of optimal pumping speed is obtained, them other parameter sets may be tested to locate an efficiency maximum. It may be useful to first vary the most sensitive factor, such as water input, to first optimize it to the choice of pumping speed, and the other factors may be varied to explore the input parameter space.

The next step would be would be to repeat the previous paragraph (for each of the remaining parameters) with the chamber volume varying first.

As noted, this process would be repeated in a factorial methodology to generate an overall data table that may be evaluated to ensure the best configuration of the five parameters to optimize the cooling available to the desired volume.

### V.C. Dual Chambers

In some cases, it may be desirable to have two chambers side-by-side, with valves to alternately connect vacuum pump 114 to one, then the other, as the two chambers cycle between some elevated pressure (perhaps just above a pressure for a desired evaporation temperature, perhaps atmospheric, perhaps some other elevated pressure) and the pressure that corresponds to the desired degree of cooling (or perhaps slightly below). While the first chamber is at the higher pressure, atomizer 120 sprays water into aluminum cylinder 410. The valve to the pump connected to the first chamber opens and the chamber is evacuated and vaporization and cooling occurs. During this pump down, the second chamber is at the elevated pressure and atomizer 120 sprays the water into the second aluminum cylinder 410. When the first cylinder vacuum is completed, its valve to the pump closes and the valve connecting the same pump to the second chamber opens and evacuates that second chamber. During this second chamber pump down and vaporization, the first chamber is vented to the elevated pressure and atomizer 120 sprays again into its aluminum cylinder. When the second chamber pump down and vaporization is completed, the approach repeats with the first chamber/cylinder.

In this way, one achieves high utilization of vacuum pump 114 and can drive more cooling to the cold room/commercial refrigeration system etc. The process will be optimized with respect to time utilization if the pump-down times are relatively short (5 to 10 seconds) so that elimination of the vent/atomization time has the largest impact on improved utilization.

When the chambers are vented, the venting may use dry nitrogen. Alternatively, the spray injection may use dry nitrogen as a dispersant gas. This may be especially desirable if the ambient air is extremely humid. Alternatively, incoming air may be run through a dryer. This ensures that there is zero water vapor in the chamber when atomizer 120 sprays and enables the absolute highest level of water vapor from the walls to be vaporized and drawn into vacuum pump 114.

### V.D. Control of Bleed Gas and Water Spray and Multi-Stage Pump

Water vapor at room temperature and atmospheric pressure (1013 mbar, 101.3 kPa) operates as a real gas, rather than an ideal gas. (An ideal gas follows the ideal gas law, PV=nRT, where P is the pressure in atmospheres, V is the volume in liters, n is the number of moles of gas, R is the gas constant [0.0821 1-atm/mole-° K], and T is the temperature in Kelvin). At atmospheric pressure, the amount of water vapor that can be contained in a volume of one liter at 30° C. is about 0.031 grams. If one reduced the pressure from atmospheric to say, 20 mbar while holding temperature constant, if the water vapor perfectly behaved Boyle's law then one would expect the amount of water vapor that could be held in a cubic meter would be reduced by a factor of 20/1013. For this example, at 30° C., one cubic meter could hold 6.12×10⁻⁴ grams.

However, when the pressure is lowered below a certain value, the intermolecular forces are attenuated by distance, and water vapor transitions to behave as an ideal gas. At 30° C., if the water vapor partial pressure is below 100 mbar (10 kPa), water vapor behaves as an ideal gas to within about 0.1%.

In vacuum chamber 102, 230 operating pressures are well below 100 mbar. In this range, the water vapor behaves as an ideal gas, in which the ideal gas law is accurate to 0.1%. As a result, for our example at 20 mbar and 30° C., the ideal gas law computes that a one-liter volume would be able to hold 0.0144 grams, or 23.5 times higher than a naïve Boyle's law calculation from room temperatures and pressures. For cooling achieved through the evaporation of water under vacuum, this increase in the amount of water that may be held in a defined volume may enable high efficiency and economical cooling. This advantage may be observed to progressively improve, as deviation from ideal gas behavior falls from 5% to 2%, to 1%, to 0.5%, or 0.2% to 0.1%.

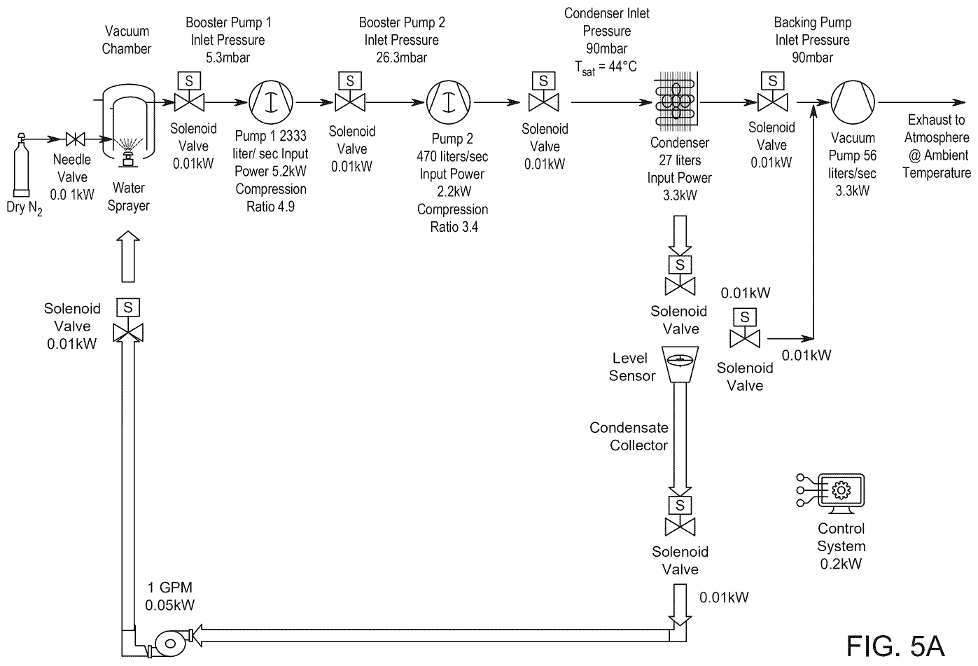

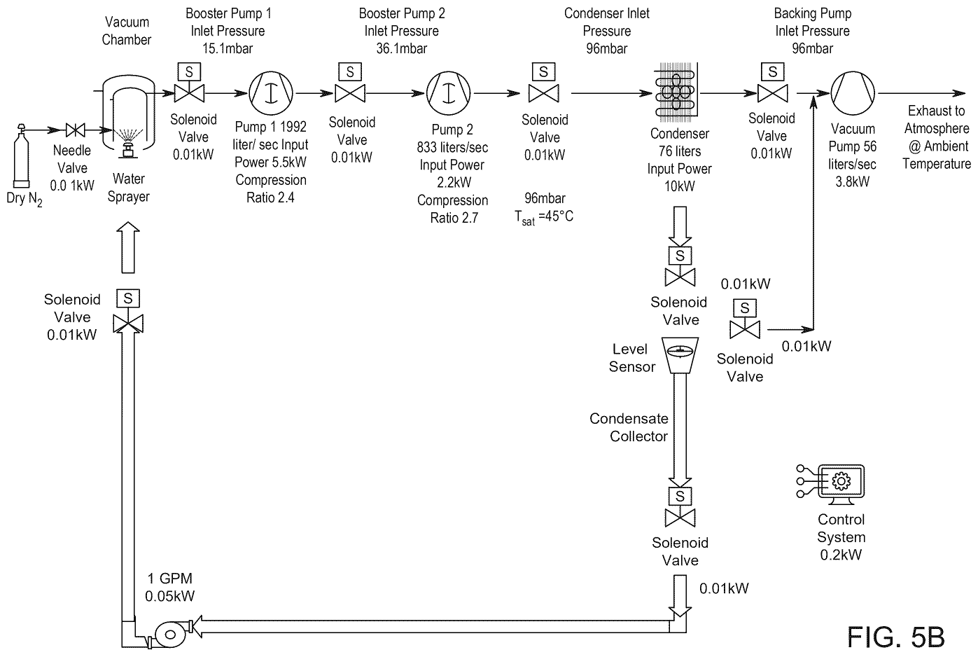

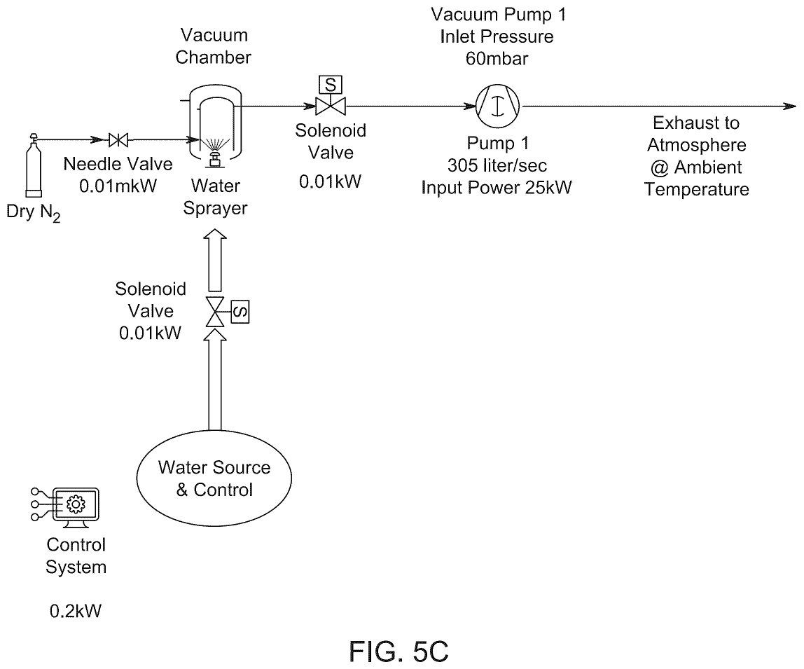

FIG. 5A shows a possible refrigeration system targeting industrial/commercial/data storage applications that require significant amounts of cooling from about 5 refrigeration tons to over 100 refrigeration tons. (One refrigeration ton is a unit of cooling power for refrigeration applications. It was originally defined as the rate of heat transfer that results in freezing or melting of 1 short ton of pure ice at 0° C. in 24 hours. It is now standardized at 12,000 Btu/h. A large window room air conditioner is about 12,000 Btu/hr. or one refrigeration ton. A typical residential air conditioning system is about three to five refrigeration tons.) The example shown in FIG. 5A targets a cooling capacity of about 5.7 refrigeration tons. A vacuum chamber is used to contain a combination of dry nitrogen (or dry air) and water droplets. The droplets will be sprayed against the walls of the vacuum chamber so that the evaporation maximizes heat flow from the walls. The system includes a source of dry nitrogen gas, a water feed system to water sprayer 120 inside the vacuum chamber, three vacuum pumps, a water vapor condenser, and several solenoid valves to isolate sections of the system from other areas. The system may also include computer control 490 that enables precise automatic (or manual) control of each of the system elements. This system utilizes the fact that at a pressure of 5.3 mbar in the vacuum chamber, the water vapor behaves as an ideal gas. The diagram shows the power consumption at each point of the refrigeration cycle. The large power consumption is the vacuum pump(s) 114 and the condenser, which total about 13 kW.

Cooling capacity may be calculated from the mass flow of water in and waver vapor out. The mass density of water vapor at 5.3 mbar is 0.0038 grams/liter. If vacuum pump 114 can pump 2000 liters/sec then the system pumps 31,916 grams (or about 32 Kg) per hour

$${H}_{2} O Mass Flow from vacuum chamber ( at 5.3 mbar ) = ( 0.0038 grams {H}_{2} O / l ) * ( 2333 l / s ) * 3 , 600 s / hr = 31 , 916 grams / hr$$

Which translates into cooling power at 2,260 J/g:

$$31 , 916 g * ( 2 , 260 J / g ) / ( 1055 J / BTU ) / ( 12 , 000 BTU / ton ) = 5.7 tons Refrigeration$$

### Yields a Coefficient of Performance (COP)=1.4

In some cases, a small amount of dry nitrogen or air may be bled into the vacuum chamber, either fed into the water stream before sprayer 120, or via a separate parallel bleed valve. The nitrogen bleed may be tuned to optimize the pumping efficiency of the vacuum pumps.

Each model of vacuum pump has a range in which it operates most efficiently. By combining multiple pumps in a multi-stage design, several pumps can be chosen, and the system can be tailored to the most-advantageous pumping range for each pump.

A first booster pump maintains vacuum chamber 102, 230 in the range of 5.3 mbar (cycling a bit above to a bit below, as discussed below), and pressurizing the water vapor to 26.3 mbar for the second booster pump. First booster pump may pump at about 2,333 liters/sec. The inflow of dry nitrogen gas and water droplets from water sprayer 120 inside into vacuum chamber 102, 230 may be controlled to maintain the pressure within the chamber at about 5.3 mbar. This pressure causes water to vaporize from the walls of the aluminum chamber at a temperature of −1.7° C. (29° F.). Further, Booster Pump 1 is selected to enable an outlet pressure from the pump of 26.3 mbar (the justification for this pressure is to enable a match with the pumping capabilities of Booster Pump 2)).

Booster Pump 2 pumps the water vapor and nitrogen from its input side of 26.3 mbar to step it up to 90 mbar. The pumping rate for Booster Pump 2 may be about 470 liters/sec (2,333 (the pumping rate of Booster Pump 1)×5.3 (the input pressure of Booster Pump 1)/263 mbar (input pressure of Booster Pump 2)=470 liters/s

Booster Pump 2 has an outlet pressure of 90 mbar. At the 90 mbar stage, a water-vapor condenser may condense water from the nitrogen gas/water vapor flow. The condenser may extract about 27 liters of liquid water per hour, which may flow back to be injected into vacuum chamber 102, 230 at water sprayer 120. The pressure of 90 mbar corresponds to a saturation temperature at 44° C. (111° F.). This saturation temperature may allow the condenser to operate effectively outdoors at all seasons of the year.

With the water vapor removed, the third stage pump—a Backing Pump—may further compress the remaining nitrogen gas and small amount of water vapor to atmospheric pressure to be exhausted to ambient. The Backing Pump may operate at about 56 liters/sec.

Each of the three pumps, and the pressures between the pumping stages, may be chosen to match “sweet spots” in the pumping capabilities of available pumps. By matching the pressures and pumping needs to optimal capabilities, the overall cost and efficiency of the system may be improved.

Computer control 490 may direct control of all of the elements in the system. The control loop may monitor sensors of the temperature of the target volume, temperature of the coolant liquid flowing to the target volume, temperature of the cylinders (inner and outer), and/or pressures of various stages, to provide feedback to computer control 490 so that computer control 490 may adjust each of the elements to achieve the desired target pressure (5.3 mbar in the vacuum chamber in the above examples). For example, computer control 490 may:
monitor the pressure within the chamber by vacuum gauge 434,
control the needle valve of the dry nitrogen gas input in conjunction with control of the water sprayer to input the required amount of gas and water droplets into the vacuum chamber to match the pumping speed of Booster Pump 1.

A target pressure of approximately 5.3 mbar may be maintained, for example, cycling between an upper level of 5.5 mbar and a lower level of 5.1 mbar. For example, when vacuum chamber 102, 230 reaches 5.5 mbar, computer control 490 may send electrical signals to increase the rate of the first booster pump, and send electrical signals to close a needle valve on the dry nitrogen feed and water droplet sprayer to reduce their inputs to the vacuum chamber (to zero if necessary). Then the first booster pump may pump the pressure down past the center set point to the lower end of the target range, for example, 5.1 mbar. Computer control 490 may then send electrical signals to the dry nitrogen needle valve and the water droplet sprayer to increase their inputs to the vacuum chamber, and may send signals to decrease the rate of the first booster pump (generally it would be undesirable to stop the pump entirely, because it takes time and energy to spin it back up to full power). This would occur in real time in an iterative process to ensure that the pressure in the vacuum chamber would be maintained at 5.3+/−0.2 mbar as in this example.

The system of FIG. 5A may be changed to provide different levels of cooling at other targeted temperatures using the same overall control process but with different vacuum pumps and water-vapor condenser.

FIG. 5B shows an overall cooling system that may be appropriate for cooling a volume to air conditioning temperatures. The system may include a source of dry nitrogen gas, a water feed system to water sprayer inside the vacuum chamber, three vacuum pumps, a water vapor condenser, and several solenoid valves to isolate sections of the system from other areas.

The target pressure in the vacuum chamber may be 15.1 mbar+/−an acceptable upper and lower ranges. At 15.1 mbar, the water droplets will vaporize at 12.8° C. (55° F.) that is suitable for air conditioning. At this pressure, the water vapor behaves as an ideal gas. Pumping rates may be chosen to evaporate 0.0109 grams per liter of water per second. The coefficient of performance of this configuration may be calculated as follows, assuming that the mass density of water at 15.1 mbar of 0.0109 grams/liter, and that the pump can pump at a rate of 1992 liters/sec

$${H}_{2} O Mass Flow from vacuum chamber ( at 1 5.1 mbar ) = ( 0.0109 grams {H}_{2} O / l ) * ( 1992 l / s ) * 3 , 600 s / hr = 78 , 289 grams / hr$$

Which translates to cooling power as follows:

$$= 78 , 289 g * ( 2 , 260 J / g ) / ( 1055 J / BTU ) / ( 12 , 000 BTU / ton ) = 14 tons AC$$

This will generate 14 tons of air conditioning at a Coefficient of Performance (a refrigeration metric) of 2.45. Similarly, the SEER value may be >15 (a high efficiency value).

The operational control would be similar to that described above for FIG. 5A but the vacuum pumps and condenser would be selected to provide a pressure within the vacuum chamber of 15.1 mbar+/−an acceptable range (for example, the acceptable pressures may range from 14.9 mbar to 15.3 mbar). An amount of dry nitrogen gas and water droplets input into the vacuum chamber may be controlled by measuring the pressure in the chamber by vacuum gauge 434 and then adjusting the flows of gas and water to maintain the pressure in the chamber at 15.1 mbar+/−an acceptable range of values around 15.1 mbar.

Another possible configuration is shown in FIG. 5C. In this configuration, the pressure in the vacuum chamber is higher than in FIG. 5A or 5B but the pressure is still in the region where water vapor behaves as an ideal gas. In this approach, the system has a source of dry nitrogen gas, a water feed system to water sprayer inside the vacuum chamber, one vacuum pump, and several solenoid valves to isolate sections of the system from other areas. In the similar manner discussed for FIGS. 5A and 5B, computer control 490 would ensure that the pressure in the vacuum chamber will be 60 mbar+/−2 mbar (in the range of 58 mbar to 62 mbar). Cooling power of the apparatus of FIG. 5 may be computed as:

$${H}_{2} O Mass Flow from vacuum chamber ( at 5.3 mbar ) = ( 0.044 grams {H}_{2} O / l ) * ( 305.6 l / s ) * 3 , 600 s / hr = 48760 grams$$

49 Kg of water vapor per hour produces cooling power calculated

$$= 48 , 760 g * ( 2 , 260 J / g ) / ( 1055 J / BTU ) / ( 12 , 000 BTU / ton ) = 8.7 tons Refrigeration$$

This would enable a significant level of cooling with a COP=1.22 with a simpler system configuration than with the systems shown in FIGS. 5A and 5B. The cooling would take place at a higher temperature than the configurations displayed in FIG. 5A or 5B (36° C.) that corresponds to the vapor pressure of the water vapor at 60 mbar.

In addition to the configurations displayed in FIGS. 5A, 5B and 5C, other configurations may meet the varying cooling and refrigeration requirements of industrial and residential systems. These configurations may employ a variety of vacuum pumps or condensers to achieve the needed cooling amounts.

### VI. EMBODIMENTS

An object or space may be cooled by placing the exterior surface of a cooling wall of a vacuum chamber against the object or space; spraying water from a mister into the vacuum chamber or against the cooling wall; maintaining vacuum in the vacuum chamber sufficient to cause accelerated evaporation of the water and cooling of the cooling wall to a temperature desired for cooling of the object.

An apparatus for cooling an object or space, may include a vacuum chamber with a cooling wall designed to be placed against the object or space to be cooled; a water spray designed to spray water into the vacuum chamber or against the cooling wall; and apparatus designed to maintain vacuum in the vacuum chamber sufficient to cause accelerated evaporation of the water and cooling of the cooling wall to a temperature desired for cooling of the object or space.

A patient may be treated by providing a vacuum chamber against tissues of the patient for which cooling is desired for treatment; spraying water from a mister into the vacuum chamber or against a cooling wall of the vacuum chamber; and maintaining vacuum in the vacuum chamber sufficient to cause accelerated evaporation of the water and cooling to a temperature desired for cooling of tissues of the patient.

Apparatus for treating a patient may include a vacuum chamber having a cooling wall designed to be placed against tissues of the patient for which cooling is desired for treatment; a water spray designed to spray water into the vacuum chamber; and maintaining vacuum in the vacuum chamber sufficient to cause accelerated evaporation of the water and cooling to a temperature desired for cooling of tissues of the patient.

A refrigeration device may include a thermally-conductive platen, possibly connected to a set of cooling fins; a vacuum bell sealed to the thermally conductive platen; a spray device mounted to spray water onto the thermally-conductive platen; a tank fluidly connected to the spray device to supply liquid to the spray device; a source of vacuum designed to cool the platen by drawing vacuum to accelerate evaporation of the water; an electronic computer programmed to obtain readings from temperature sensors, and based on those readings, to provide control signals to the water spray and to control vacuum pressure to achieve a level of evaporative cooling at the platen effective to induce a desired cooling of the platen; a device, such as a fan, to direct a flow of air across the platen and cooling fins; to induce a cooling of a selected region within the refrigeration device; a condenser to convert the coolant from vapor phase to liquid phase; a fluid connection from the source of vacuum to the condenser; a fluid connection from the condenser to the tank that supplies liquid to the spray device.

A device for medical or other therapeutic cooling of selected regions of a patient using vacuum-induced evaporative cooling of a liquid medium may include a conformal, highly thermal conductive platen for thermal contact with a region of tissue that is desired to be cooled, coated with a thin layer of a non-metallic release material to prevent sticking by freezing: a vacuum bell sealed to the thermally conductive platen; a vacuum seal between the chamber and the thermally conductive platen; a spray device mounted to spray water into the chamber onto the thermally-conductive platen; a source of vacuum connected to the chamber designed to cool the platen by drawing vacuum to accelerate evaporation of the water; an air inlet; a three-way valve assembly between vacuum pump 114 and air inlet to the chamber; a set of thermal sensors mounted within the thermally conductive platen; an exhaust of the vapor to the exterior of the desired cooled region; a device to generate a column of air across the conductive platen; an electronic computer programmed to obtain readings from temperature sensors, and based on those readings, to provide control signals to the water spray and to control vacuum pressure to achieve a level of evaporative cooling at the platen effective to induce a therapeutic result in a patient.

An air-conditioning device may include: a thermally-conductive platen connected to a set of cooling fins; a vacuum bell sealed to the thermally conductive platen; a spray device mounted to spray water onto the thermally-conductive platen; a tank fluidly connected to the spray device to supply liquid to the spray device; a source of vacuum designed to cool the platen by drawing vacuum to accelerate evaporation of the water; an electronic computer programmed to obtain readings from temperature sensors, and based on those readings, to provide control signals to the water spray and to control vacuum pressure to achieve a level of evaporative cooling at the platen effective to induce a desired cooling of the platen; a device, such as a fan, to direct a flow of air across the platen and cooling fins; to induce a cooling of a selected region of a room or enclosure; a condenser to convert the coolant from vapor phase to liquid phase; a fluid connection from the source of vacuum to the condenser; and a fluid connection from the condenser to the tank that supplies liquid to the spray device.

A method for cooling of selected regions of an enclosure using vacuum-induced evaporative cooling of a liquid medium may include: placing a highly thermally-conductive platen of a vacuum chamber against the region, the vacuum chamber having a vacuum bell connected to the thermally conductive platen, with a vacuum seal between the chamber and the thermally conductive platen; spraying water onto the platen via a spray device facing into the chamber; applying vacuum to the chamber, and exhausting the vapor to the exterior of the desired cooled region; receiving temperature readings from a set of thermal sensors mounted within the thermally conductive platen; generating a column of air across the conductive platen.

Specific instances may include the following features, singly or in any combination. The vacuum chamber may be formed as a vacuum bell sealed against flesh of the patient. The vacuum chamber may be formed as an enclosed volume having a cooling wall at one side, and the cooling wall is placed in physical contact with the tissues of the patient.

The conductive platen may be aluminum. The non-metallic material in contact with the tissue may prevent adhesion and tissue damage during the cooling process. The spray device may be mounted within a chamber attached to a thermally conductive material. The liquid medium may be water. The liquid medium may be saline. The liquid medium may be sodium chloride solution. The liquid medium may be calcium chloride solution. The solution level may be chosen to provide a selected liquid freezing temperature. The vacuum within the chamber may induce vaporization of the liquid from the surface of the conductive material. The vacuum application may be controlled by a valve assembly. The target temperature may be chosen to disrupt lipid-rich cells. The target temperature may be chosen to not damage the tissue skin surface. The cooling may be selected to provide analgesic effects to a desired region of the body. The cooling may be selected to ablate goblet cells. The cooling may be selected to restore normal epithelial cells. The cooling may be selected to freeze undesired gastrointestinal cells. The cooling may be selected to ablate undesired benign cells. The cooling may be selected to ablate malignant cells. The cooling may be selected to restore normal gastrointestinal cells. The cooling may be selected to provide skin lightening, i.e., hypopigmentation, to desired regions of the body. Electrical power for the system may be provided by the use solar energy converted to electricity by solar cells.

For clarity of explanation, the above description has focused on a representative sample of all possible embodiments, a sample that teaches the principles of the invention and conveys the best mode contemplated for carrying it out. The invention is not limited to the described embodiments. Well known features may not have been described in detail to avoid unnecessarily obscuring the principles relevant to the claimed invention. Throughout this application and its associated file history, when the term “invention” is used, it refers to the entire collection of ideas and principles described; in contrast, the formal definition of the exclusive protected property right is set forth in the claims, which exclusively control. The description has not attempted to exhaustively enumerate all possible variations. Other undescribed variations or modifications may be possible. Where multiple alternative embodiments are described, in many cases it will be possible to combine elements of different embodiments, or to combine elements of the embodiments described here with other modifications or variations that are not expressly described. A list of items does not imply that any or all of the items are mutually exclusive, nor that any or all of the items are comprehensive of any category, unless expressly specified otherwise. In many cases, one feature or group of features may be used separately from the entire apparatus or methods described. Many of those undescribed alternatives, variations, modifications, and equivalents are within the literal scope of the following claims, and others are equivalent. The claims may be practiced without some or all of the specific details described in the specification. In many cases, method steps described in this specification can be performed in different orders than that presented in this specification, or in parallel rather than sequentially, or in different computers of a computer network, rather than all on a single computer.

The invention claimed is:

1. An apparatus for cooling a tissue, object, or space, comprising:
a vacuum chamber designed to be placed in thermal contact with the tissue, object, or space to be cooled;
a water sprayer designed to spray water as a coolant into the vacuum chamber or against a cooling wall of the vacuum chamber, the sprayer designed for use of water as coolant;
a vacuum pump and computer control designed to maintain vacuum in the vacuum chamber, the maintained vacuum being below ambient pressure and sufficient to cause accelerated evaporation of the water and cooling to a temperature for cooling of the tissue, object, or space.

2. The apparatus of claim 1:
the cooling wall of the vacuum chamber being a cylinder with a vertical axis, the water sprayer being located on the cylinder axis.

3. The apparatus of claim 2, further comprising:
an outer cylinder surrounding and concentric with the cooling wall cylinder, a space between the outer cylinder and cooling wall cylinder being designed as a water jacket, designed for flow of water between the cylinders to carry cooling to the tissue, object, or space by convection.

4. The apparatus of claim 2, the computer control being further programmed to cycle a water pump and/or the vacuum pump to maintain a temperature for cooling of the tissue, object, or space.

5. The apparatus of claim 2, further comprising:
a conical chamber between the water sprayer and the cylindrical cooling wall cylinder.

6. The apparatus of claim 2, further comprising:
a chamber in gas communication with the cylindrical vacuum chamber, being of volume to contain air to dissolve water vapor.

7. The apparatus of claim 2:
the cooling wall cylinder being formed of aluminum or an aluminum alloy.

8. The apparatus of claim 2:
the cooling wall cylinder being roughened or otherwise treated to increase surface tension with droplets of cooling water.

9. The apparatus of claim 1, further comprising:
an enclosure surrounding the vacuum chamber, a space between the outer enclosure and vacuum chamber being designed as a water jacket, designed for flow of water through a space between the enclosure and vacuum chamber carry to carry cooling to the tissue, object, or space by convection.

10. The apparatus of claim 9, the computer control being further programmed to cycle a water pump and/or the vacuum pump to maintain a temperature for cooling of the tissue, object, or space.

11. The apparatus of claim 1, the computer control being further programmed to cycle a water pump and/or the vacuum pump to maintain a temperature for cooling of the tissue, object, or space.

12. The apparatus of claim 11:
the cooling wall of the vacuum chamber being a cylinder, the water sprayer being located on the axis of the cylinder.

13. The apparatus of claim 11, the computer control being further programmed to cycle the vacuum pump on-off or to cycle speed of the vacuum pump to reduce icing.

14. The apparatus of claim 11, the computer control being further programmed to cycle the vacuum pump on-off or to cycle speed of the vacuum pump to maintain pressure in the vacuum chamber near the pressure corresponding to the temperature for cooling of the tissue, object, or space.

15. The apparatus of claim 11, the computer control being further programmed with control parameters optimized using multi-parameter factorial experiment optimization.

16. The apparatus of claim 1:
further comprising a second vacuum chamber;
the computer control being further programmed to cycle draw of the vacuum pump between the two chambers.

17. The apparatus of claim 1, further comprising:
a vent into a region, designed to add humidity to the region.

18. The apparatus of claim 1, further comprising:
one or more sensors designed to generate signals to indicate state of a variable within the apparatus;
the computer control further designed to receive the signals and from the signals to compute an icing condition, and based on the icing computation, to control the apparatus to relieve the icing condition.

19. The apparatus of claim 1, wherein:
the vacuum chamber is formed as an open vacuum bell sealed against flesh of a patient.

20. The apparatus of claim 1, wherein:
the vacuum chamber is formed as an enclosed volume having a cooling wall at one side, and the cooling wall is placed in physical contact with tissues of a patient.

21. The apparatus of claim 1, wherein:
the vacuum chamber is formed as a vacuum bell sealed to a flat thermally conductive platen, the platen designed to be placed in thermal contact with the tissue, object, or space.

22. The apparatus of claim 21, wherein:
the platen is coated with a non-metallic nonstick release material designed to prevent adhesion and resultant tissue damage resulting from freezing of the tissue to be cooled or water between the tissue and the platen.

23. The apparatus of claim 1, wherein:
the water has in solution an electrolyte chosen to depress freezing point of the water to a desired temperature.

24. The apparatus of claim 1, wherein:
the computer control is programmed to provide cooling of the tissue designed to disrupt adipose tissue.

25. The apparatus of claim 1, wherein:
the computer control is programmed to provide cooling of the tissue designed to reduce pain.

26. The apparatus of claim 1, wherein:
the computer control is programmed to provide cooling of the tissue designed to lighten skin and/or to reduce hypopigmentation.

27. A method for cooling a tissue, object, or space, comprising:
placing a vacuum chamber in thermal contact with the tissue, object, or space to be cooled;
spraying water from a sprayer into the vacuum chamber or against a cooling wall of the vacuum chamber, the sprayer designed for use of water as coolant;
via computer control, maintaining vacuum in the vacuum chamber, the maintained vacuum being below ambient pressure and sufficient to cause accelerated evaporation of the water and cooling to a temperature desired for cooling of the tissue, object, or space.

28. The method of claim 27, further comprising:
at the computer control, receiving signals from one or more sensors designed to indicate state of a variable within the apparatus;
at the computer control, computing an icing condition, and based on the icing computation, to control the apparatus to relieve the icing condition.

29. The method of claim 27, wherein:
the tissue to be cooled is in the gastrointestinal tract.

30. The method of claim 27, wherein:
the tissue to be cooled is in the respiratory tract.

31. The method claim 27, wherein:
the tissue to be cooled includes goblet cells to be disrupted.

32. The method of claim 27, wherein:
the tissue to be cooled includes malignant cells to be ablated.

33. The method of claim 27, wherein:
the tissue to be cooled includes undesired benign cells that are selectively sensitive to cold, and the cooling is designed to disrupt these undesired benign cells.
